SEQUENCE LISTING (35 sequences)

SEQ ID NO: 1 - DNA, 28 bp, Artificial
  primer
ggccggtaccatgcaccatgtgtttaaa

SEQ ID NO: 2 - DNA, 28 bp, Artificial
  primer
ggccgatatcctccacgttcttgtactt

SEQ ID NO: 3 - DNA, 29 bp, Artificial
  primer
ggccccgcggtcactccacgttcttgtac

SEQ ID NO: 4 - DNA, 29 bp, Artificial
  primer
ggccccgcggtcactccacgttcttgtac

SEQ ID NO: 5 - DNA, 29 bp, Artificial
  primer
ggccgagctcatgcaccatgtgtttaaag

SEQ ID NO: 6 - DNA, 26 bp, Artificial
  primer
ggccccgcggcgttacatgctcagga

SEQ ID NO: 7 - DNA, 28 bp, Artificial
  primer
ggccgagctctccagccacatctcggag

SEQ ID NO: 8 - DNA, 29 bp, Artificial
  primer
ggccccgcggtcactccacgttcttgtac

SEQ ID NO: 9 - DNA, 21 bp, Artificial
  primer
tttggaggtgacggaactgga

SEQ ID NO: 10 - DNA, 21 bp, Artificial
  primer
tgaaggcctgtgatcgctaac

SEQ ID NO: 11 - DNA, 21 bp, Artificial
  primer
agatgtctcgctccgtggcct

SEQ ID NO: 12 - DNA, 23 bp, Artificial
  primer
cccacttaactatcttgggctgt

SEQ ID NO: 13 - DNA, 19 bp, Artificial
  primer
catccgccttcctgagcat

SEQ ID NO: 14 - DNA, 18 bp, Artificial
  primer
agcaggccttcccgtttc

SEQ ID NO: 15 - DNA, 22 bp, Artificial
  primer
tgaatttggaggtgacggaact

SEQ ID NO: 16 - DNA, 20 bp, Artificial
  primer
cagaaggcccatgagcagaa

SEQ ID NO: 17 - DNA, 18 bp, Artificial
  primer
cctggcacccagcacaat

SEQ ID NO: 18 - DNA, 20 bp, Artificial
  primer
gccgatccacacggagtact

SEQ ID NO: 19 - DNA, 24 bp, Artificial
  primer
atgaagaaggacgagtcgttcctg

SEQ ID NO: 20 - DNA, 27 bp, Artificial
  primer
tcactccacgttcttgtacttggtgaa

SEQ ID NO: 21 - DNA, 21 bp, Artificial
  primer
atgtcctccgcgccgcgctcg

SEQ ID NO: 22 - DNA, 27 bp, Artificial
  primer
tcactccacgttcttgtacttggtgaa

SEQ ID NO: 23 - DNA, 27 bp, Artificial
  primer
atgcaccatgtgtttaaagatcaccaa

SEQ ID NO: 24 - DNA, 27 bp, Artificial
  primer
tcactccacgttcttgtacttggtgaa

SEQ ID NO: 25 - DNA, 29 bp, Artificial
  primer
tcgaattcaatggccacctccccgcagaa

SEQ ID NO: 26 - DNA, 30 bp, Artificial
  primer
tgctctagatcactccacgttacggtactt

SEQ ID NO: 27 - DNA, 19 bp, Artificial
  primer
gggacctgactgactacct

SEQ ID NO: 28 - DNA, 20 bp, Artificial
  primer
ctagaagcatttgcggtgga

SEQ ID NO: 29 - protein, 397 aa, Homo sapiens [sequence omitted]

SEQ ID NO: 30 - protein, 50 aa, Homo sapiens
MHHVFKDHQRGEKRGFLSPEKKNCRRLELRRGCSCSRGLCSQALMASLAG

SEQ ID NO: 31 - protein, 37 aa, Homo sapiens
MSSAPRSPTPRPRRMKKDESFLGKLGGTLARKRRARE

SEQ ID NO: 32 - protein, 22 aa, Homo sapiens
KKDESFLGKLGGTLARKRRARE

SEQ ID NO: 33 - protein, 50 aa, Homo sapiens
SLLPGSDRSGVETSEYAQGGVSDLQEEGKNAINSPMSPALADVHPEDTQL

SEQ ID NO: 34 - protein, 30 aa, Homo sapiens
VSDLQEEGKNAINSPMSPALADVHPEDTQL

SEQ ID NO: 35 - protein, 30 aa, Homo sapiens; identical to SEQ ID NO: 34

(19) United States
(12) Patent Application Publication
(10) Pub. No.: US 2006/0211008 A1
(43) Pub. Date: Sep. 21, 2006

(54) Beta-parvin expression for use in diagnostic methods for assessment of breast cancer

(76) Inventors: Perry Mongroo, Etobicoke (CA); Greg Hannigan, Toronto (CA)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/232,593

(22) Filed: Sep. 22, 2005

Related U.S. Application Data
(60) Provisional application No. 60/611,693, filed on Sep. 22, 2004.

(51) Int. Cl. C12Q 1/68

(52) U.S. Cl. 435/6

(57) ABSTRACT

The invention is directed to polynucleotides encoding β-parvin, a newly identified tumor suppressor gene the alteration of which is involved in the occurrence of breast cancer in a patient, as well as to corresponding expressed polypeptides. The invention also concerns diagnostic methods using the β-parvin polynucleotides and/or peptides and embodiments thereof as diagnostic or therapeutic tools for breast cancer or in assays to identify pharmacological agents for the treatment of breast cancer.

### FIELD OF THE INVENTION

The present invention pertains to polynucleotides useful as diagnostic tools for predicting the occurrence and prognosis of breast cancer. More specifically, the invention is directed to polynucleotides encoding β-parvin, a newly identified tumor suppressor gene the alteration of which is involved in the occurrence of breast cancer in a patient, as well as to corresponding expressed polypeptides. The invention also concerns diagnostic methods using the β-parvin polynucleotides and/or peptides and embodiments thereof as diagnostic or therapeutic tools for breast cancer or in assays to identify pharmacological agents for the treatment of breast cancer.

### BACKGROUND OF THE INVENTION

ParvA (α-parvin) and ParvB (β-parvin) are ubiquitously expressed members of a recently identified family of adaptor proteins, which have been implicated in integrin-mediated cell adhesion through binding to the integrin linked kinase (ILK). ParvA has also been called actopaxin or CH-ILKBP1, and ParvB is also known as affixin (Nikolopoulos & Turner, 2001; Tu et al., 2001; Yamaji et al., 2001). Interactions of ParvA or ParvB with ILK are mediated by one of two conserved calponin homology (CH) domains, a structural motif that also mediates protein interactions with actin (Banuelos et al., 1998; Gimona & Winder, 1998). The C. elegans Parvin/Pat-6 homologue intracts genetically with ILK/Pat-4, and is essential for assembly of integrin/Pat-3-dependent muscle attachment structures (Lin et al., 2003; Mackinnon et al., 2002), which are analogous to focal adhesions of mammalian cells (Hresko et al., 1994; Williams & Waterston, 1994). In worms, parvin/Pat-6 is an essential protein which functionally links the actin cytoskeleton to integrin-mediated cell adhesion.

ParvA and ParvB are encoded by different genes in humans, and their highly conserved intron-exon structure suggests they arose from a relatively recent gene duplication event. The human ParvA gene maps to chromosome 11p15.5, and ParvB maps to chromosome 22q13.31 (Korenbaum et al., 2001; Olski et al., 2001). Regulation of ParvB expression is complex, and alternative transcripts involving differential 5′ and 3′ splicing have been identified (Korenbaum et al., 2001; Olski et al., 2001). The biological significance of different ParvB proteins is unknown, however, all known isoforms retain the tandem arrangement of CH1 and CH2 domains, thus, are all likely capable of binding to ILK. BLAST searches (Altschul et al., 1997) indicate C. elegans and Drosophila melanogaster each have a single parvin gene, implying the evolution of distinct functions of ParvA and ParvB in mammals. In addition to modulating cell adhesion, ParvA potentiates ILK signaling (Attwell et al., 2003; Fukuda et al., 2003), Microdeletions nearby the ParvB locus on chromosome 22q13.31 are associated with colon and breast cancers (Castells et al., 2000).

Two key regulatory targets of ILK signaling are the serine/threonine (S/T) kinases, PKB and glycogen synthase kinase 3β(GSK3β) (Delcommenne et al., 1998), both of which regulate cell proliferation and apoptosis (Coffer et al., 1998; Kim & Kimmel, 2000). ILK-mediated phosphorylation of PKB on S473 activates PKB and increases cell survival (Attwell et al., 2000), while phosphorylation of GSK3β on S9 inhibits its activity, thereby promoting cell proliferation through stabilization of β-catenin and cyclin D1 (D'Amico et al., 2000; Radeva et al., 1997). In the MMTV-ILK transgenic mouse model of mammary carcinoma, tumors display increased levels of PKB pSer473 and GSK3β pSer9, as well as increased cyclin D1 expression (White et al., 2001). Silencing of either ILK or ParvA expression inhibits PKB S473 and GSK3β S9 phosphorylations, and thus, ParvA appears to play a role in promoting ILK signaling of these downstream kinases (Attwell et al., 2003; Fukuda et al., 2003).

Inhibition of cellular ILK activity by small molecules or dominant negative mutants suppresses the transformed phenotype (Persad & Dedhar, 2003), stimulating interest in ILK as a target for anti-cancer drug development (Edwards et al., 2004). Physiologic inhibitors of ILK signaling include PTEN, a lipid phosphatase that is the major antagonist of PI3K activity (Leslie & Downes, 2002; Maehama & Dixon, 1999; Mills et al., 2001; Myers et al., 1998). Dephosphorylation of PI(3,4,5)P₃ by PTEN indirectly inhibits ILK activity, which is accordingly elevated in PTEN null cells (Persad et al., 2001). Recently discovered is ILKAP, a serine/threonine phosphatase that selectively binds to and inhibits ILK, blocking phosphorylation of GSK3 S9 and suppressing downstream transactivation by β-catenin/TCF factors (Leung-Hagesteijn et al., 2001).

It is now demonstrated that the expression of ParvB inhibits ILK signaling and suppresses cellular transformation in breast cancer. ParvB is now demonstrated to be downregulated in advanced breast cancers identifying its function as a tumor suppressor gene.

### SUMMARY OF THE INVENTION

ParvB has now been characterized as a tumor suppressor gene that when down regulated or not expressed in breast tissue leads to oncogenic signaling via ILK and the development of breast cancer. ParvB expression is down-regulated in breast tumors, relative to patient-matched normal mammary gland tissue. ParvB protein levels are reduced by ≧90% in advanced tumors, relative to matched normal breast tissue. Conversely, ILK protein and kinase activity levels are elevated in these tumors, suggesting that downregulation of ParvB stimulates ILK signaling. Only very low levels of ParvB mRNA and/or protein are detected in MDA-MB-231 and MCF7 breast cancer cells. Overall these results demonstrate that loss of ParvB expression is a novel mechanism for upregulating ILK activity leading to the development or progression of breast cancers.

With the knowledge that ParvB is a tumor suppressor gene implicated in the development of breast cancer, various diagnostic, prognostic and therapeutic uses of the ParvB nucleotide and amino acid sequences can be used.

According to an aspect of the invention is a nucleic acid sequence encoding a ParvB tumor suppressor protein. According to a further aspect of the invention is an amino acid sequence encoding a ParvB tumor suppressor protein.

In aspects of the invention, the ParvB nucleotide sequence can be selected from any one of the ParvB1-3 cDNA isoforms encoding a ParvB protein of 350, 364 and 397 amino acids, respectively. In further aspects, the ParvB3 nucleotide sequence is selected for use in the present invention.

According to an aspect of the present invention is the use of a nucleic acid encoding a ParvB tumor suppressor gene product, or a functional fragment, variant or fusion product thereof according to the invention, or a nucleic acid with at least 60%, preferably 70%, more. preferably 80%, most preferably 90% sequence identity to said nucleic acid as measured by a BLASTN search (Altschul et al., 1997), or a functional fragment thereof in diagnosis of cancer and/or prediction of the likelihood of developing cancer. In an embodiment of the invention is the use of said nucleic acid, whereby said cancer is breast cancer.

Diagnosis and/or prediction can be based on the detection of mutations, comprising point mutations, deletions, insertions and rearrangements, in the ParvB tumor suppressor gene or located on chromosome 22q13.31, and/or by measuring the transcription level of the ParvB tumor suppressor gene or any of the different ParvB transcripts for the various ParvB isoforms (i.e. ParvB1-3). This analysis can be performed by techniques such as, as a non-limiting example, DNA/DNA hybridization, DNA/RNA hybridization, fluorescent in situ hybridization (FISH) or PCR reaction, all known to the person skilled in the art.

Another aspect of the invention is the use of a nucleic acid encoding a ParvB tumor suppressor gene product or a functional fragment or variant thereof, according to the invention, or a nucleic acid with at least 60%, preferably 70%, more preferably 80%, most preferably 90% sequence identity to said nucleic acid as measured by a BLASTN search (Altschul et al., 1997), or a functional fragment thereof in the treatment of cancer. In one embodiment is the use of said ParvB nucleic acid in gene therapy, to restore the defective function of the tumor suppressor gene. Vectors for gene therapy are known to the person skilled in the art, and include, but are not limited, retroviral vectors, adenovirus-associated vectors and lentiviral vectors. Suitable vector systems have been described, amongst others, in WO9822143, WO9812338 and WO9817816 (the disclosures of which are incorporated herein by reference in their entirety). A preferred embodiment is said use, whereby said cancer is breast cancer.

Still another aspect of the invention is the use of a ParvB tumor suppressor gene product, or a functional fragment or variant thereof, according to the invention, or a protein with at least 60% identity, preferably 70% identity, more preferably 80% identity, most preferably 90% sequence identity to said ParvB tumor suppressor gene product, as measured by a BLASTP or TBLASTN search (Altschul et al., 1997) for the manufacture of a medicament to treat cancer. In an embodiment of the invention the cancer is breast cancer.

Still another aspect of the invention is a method to produce antibodies, using a ParvB tumor suppressor gene product or a functional fragment or variant or fusion protein thereof, according to the invention, or a protein with at least 60% identity, preferably 70% identity, more preferably 80% identity, most preferably 90% sequence identity to said ParvB tumor suppressor gene product, as measured by a BLASTP or TBLASTN search (Altschul et al., 1997), or using nucleic acid encoding such ParvB tumor suppressor gene product or a functional fragment or variant or fusion protein thereof, according to the invention, or a protein with at least 60% identity, preferably 70% identity, more preferably 80% identity, most preferably 90% sequence identity to said tumor suppressor, as measured by a BLASTP or TBLASTN search (Altschul et al., 1997). Antibodies include polyclonal, monoclonal and synthetic antibodies as is understood by one of skill in the art. Methods to produce such antibodies are known to the person skilled in the art. A further aspect of the invention is a ParvB antibody obtainable by such methods.

Still a further aspect of the invention is the use of said antibody in diagnosis of cancer and/or prediction of likelihood of developing cancer. In an aspect of the invention for said use, the cancer is breast cancer. The antibody may be used in assays such as, but not limited to, Western blot or ELISA, known to the person skilled in the art.

In accordance with a further aspect of the invention, a method is provided for producing antibodies which selectively bind to a ParvB protein comprising the steps of
administering an immunogenically effective amount of a ParvB immunogen to an animal and allowing the animal to produce antibodies to the immunogen; and
obtaining the antibodies from the animal or from a cell culture derived therefrom.

In aspects, the ParvB immunogen may be selected from any one of ParvB transcripts ParvB1-3 and may also be a fragment thereof of any one of ParvB1, ParvB2 or ParvB3 transcripts.

In accordance with a further aspect of the invention, a substantially pure antibody is provided which binds selectively to an antigenic determinant of a ParvB protein selected from the group consisting of ParvB1, ParvB2 and ParvB3.

Another aspect of the invention is use of a ParvB tumor suppressor gene product, or a functional fragment, variant or fusion product thereof, according to the invention, or a protein with at least 60% identity, preferably 70% identity, more preferably 80% identity, most preferably 90% sequence identity to said tumor suppressor, as measured by a BLASTP or TBLASTN search (Altschul et al., 1997), for the isolation of an interacting compound. Several methods have been described to detect protein-compound interactions and to select the interacting compound. These methods include, but are not limited to, phage display, yeast two-hybrid assay, co-immunoprecipitation, DNase protection assay, electrophoretic mobility shift assay, or mass spectrometric analyses, all known to the person skilled in the art, as well as fluorescence resonance energy transfer (FRET, WO99/18124 the disclosure of which is incorporated herein by reference in it's entirety) and bioluminescence resonance energy transfer (BRET, WO99/66324 the disclosure of which is incorporated herein by reference in it's entirety).

According to a further aspect of the invention is a method for detecting a nucleic acid molecule of tumor suppressor gene ParvB, comprising incubating a sample (e.g. cell lysates, cell culture, etc.) with the isolated nucleic acid molecule according to the invention and determining hybridization under stringent conditions of said isolated nucleic acid molecule to a target nucleic acid molecule as a determination of presence of a nucleic acid molecule which is the ParvB tumor suppressor gene. Such detection may be done relative to a control sample and can be used to diagnose breast cancer or for the prognosis of breast cancer.

According to a further aspect of the invention is a method for detecting a ParvB nucleotide sequence in a sample, the method comprising;
(a) providing a biological sample from a subject;
(b) adding a probe or primer capable of hybridizing with stringency to said ParvB nucleotide sequence; and
(c) detecting hybridization of said probe or primer and thus the presence of a ParvB nucleotide sequence in said sample.

According to still a further aspect of the invention is a method for treating a subject suffering from breast cancer, which method comprises administering to the subject a vector that expresses a ParvB protein effective to reduce or arrest the breast cancer. The vector may be targeted to provide intratumoral therapy.

According to yet a further aspect of the invention is a pharmaceutical composition for treating a subject suffering from breast cancer, the composition comprising a vector expressing a ParvB protein and a pharmaceutically acceptable carrier or diluent.

In accordance with a further aspect of the invention, a method is provided for suppressing the neoplastic phenotype of a breast mammary cell comprising administering to the cell an agent selected from the group consisting of
a nucleotide sequence encoding ParvB protein;
ParvB protein, fragments, polypeptides and derivatives of the polypeptides; and
an agent to stabilize ParvB protein.

In accordance with a further aspect of the invention, a method is provided for identifying compounds modulating expression of a ParvB gene comprising:
contacting a cell with a test candidate wherein the cell includes a regulatory region of a ParvB gene operably joined to a coding region; and
detecting a change in expression of the coding region.

In accordance with a further aspect of the invention, a method is provided for treating a subject having a non-transcribing or mutant ParvB gene or a lack of ParvB gene, comprising administering to the subject a therapeutically effective amount of an agent selected from the group consisting of:
(a) an isolated nucleotide sequence encoding a normal ParvB protein; and
(b) a substantially pure normal ParvB protein.

In accordance with a further aspect of the invention, a method is provided for screening for an agent useful in treating a breast cancer characterized by an increase in ILK activity, comprising screening potential agents for ability to promote the interaction as an indication of a useful agent.

According to another aspect of the invention is a method to inhibit or reduce ILK activity in a cell, the method comprising up-regulating the expression of ParvB in said cell. In aspects, the ParvB is ParvB3.

According to another aspect of the invention is a method to inhibit or reduce PKB and glycogen synthase kinase 3β phosphorylation in a cell or tissue, the method comprising upregulating the expression of ParvB in said cell or tissue.

According to another aspect of the invention is a method to suppress the cancerous transformed phenotype associated with increased ILK activity or non-regulated ILK activity in a cell, the method comprising upregulating the expression of ParvB in said cell. In aspects, the ParvB is ParvB1-3 and in more specific aspects, the ParvB is ParvB3.

According to still a further aspect of the invention is a kit for identifying an agent that modulates ParvB expression, said kit comprising a vector encoding ParvB or an analogue or fragment thereof. In aspects, the kit may comprise a vector encoding any one of ParvB1-3 isoforms as well as fragments thereof.

According to yet a further aspect of the invention is a method to identify an agent that can upregulate ParvB expression in a cell, the method comprising:
determining ParvB expression levels in a cell culture;
administering an agent to said cell culture; and
determining whether said agent has effected the ParvB expression in said cell culture. In further aspects of the method, ILK activity may be determined and the effect of the agent on the activity of ILK can be determined.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1 shows ParvB genomic organization, and expression of different isoforms in human cells. FIG. 1A. Structure of ParvB gene locus at chromosome 22q13.2/13.3 is indicated schematically. Novel ParvB3 exons 1 and 2 are indicated. The Genethon marker, D22S1171, delineates the centromeric boundary of deletions associated with breast cancers (Castells et al., 2000). Genomic clones are indicated above gene schematic, with clone sizes (kilobasepairs) in brackets. Exon 14 is untranslated. FIG. 1B. Amino acid residues 1-70 of ParvB3 are encoded by exons 1 and 2. Sequence alignment was performed using Gene Inspector v. 1.5.11 (Textco) DNA analysis software. FIG. 1C. ParvB3 specific RT-PCR was used to amplify total RNA (1 μg) isolated from HEK293, RMS rhabdomyosarcoma, and PC3 prostate carcinoma cells.

FIG. 2 shows that ParvB mRNA transcipt levels are reduced in human breast tumors and cell lines. FIG. 2A. Quantitative, real-time RT-PCR of ParvB expression in paired human breast tumors and adjacent normal mammary gland (see Materials and Methods). Error bars indicate standard deviation for triplicate determinations. Pairwise t-test across nine samples indicated significant reduction of ParvB transcript levels (P=0.005) FIG. 2B. RT-PCR analysis of ParvB expression was performed for human mammary gland and breast cell lines, using primers spanning the terminal four exons (10-13) on the 3′ end of the PARVB cDNA (pan-ParvB). All RT water control samples revealed no PCR product. FIG. 2C shows expression of ParvB mRNA in 1 DCIS, 2 lobular and 15 ductal cancers. FIG. 2D shows the expression of ParvB mRNA in 15 ductal tumors.

FIG. 3 shows the expression of ParvB3 in transfected breast cell lines. FIG. 3A. RT-PCR analysis of total RNA (1 μg) isolated from MDA/ParvB3, MCF/ParvB transfectants and vector control cells, using ParvB3 or PARVA specific primers. FIG. 3B. RT-PCR was performed on ParvB3 transfected cells, using ParvB1 or ParvB2 specific primers.

FIG. 4 shows the expression of endogenous and recombinant ParvB protein. FIG. 4A. To determine specificity, affinity purified ParvB antibody (raised against recombinant ParvB3 residues 1-142, including the common CH1 domain) was incubated with or recombinant ParvB3 or GST protein, then used to probe 50 μg HEK293 cell lysates resolved on 12% SDS-PAGE. The anti-ParvB antibody specifically detected two endogenous protein bands (45 and 40 kDa) that can be absorbed by ParvB3-GST fusion peptide. FIG. 4B. Cellular proteins (50 μg/lane) from parental and EGFP-ParvB3 HEK293 transient transfectants were resolved by 12% SDS-PAGE. Affinity purified ParvB antibody detected the exogenous ParvB3 and CH1 EGFP proteins, in addition to the endogenous ParvB protein bands. The membranes were subsequently probed with GFP and GAPDH antibodies. FIG. 4C. Domain structure of ParvB3. Full-length ParvB3 cDNA (encoding a.a. residues 1-397) and two truncated variants, CH 1 and CH 2, corresponding to residues 1-142 and 157-397 respectively were cloned into pEGFP-C3 vector as described in the examples.

FIG. 5 shows ParvB protein expression is down regulated in human breast tumors and breast cancer cell lines. Whole-cell lysates (50 μg/lane) of (A) human cell lines and (B) paired normal and tumor breast tissue lysates, were subjected to 12% SDS-PAGE and immunoblotted with ParvB antibody. Blots were then sequentially stripped and reprobed with ILK and GAPDH antibodies. Values represent ratios of tumor to normal ParvB levels, determined by densitometry of ParvB doublet bands, each internally normalized against GAPDH signal (see Materials and Methods). FIG. 5C. Normal and tumor breast tissue lysates (700 μg each) were analyzed in an ILK immune complex kinase assay using MBP as exogenous substrate. FIG. 5D a total of seven patient matched tumor/normal mammary gland samples were analyzed for ParvB protein levels. Results are presented as relative levels for each patient sample, normalized to GAPDH expression, by densitometry. The three left most samples are derived from the blots in (B).

FIG. 6 shows ParvB expression suppresses anchorage independent cell growth. FIG. 6A. ParvB3 recombinant protein expression, in MDA-MB-231 and MCF7 cells stably expressing ParvB3. Cell lysates (50 μg/lane) were analyzed by western blotting with ParvB antibody. Membranes were then stripped and reprobed with anti-GAPDH antibodies. FIG. 6B. Stable vector and ParvB cells (5×10³/well) were incubated in soft agar for 13 days at 37° C. Colonies were counted in five randomly selected fields (10x objective) per well. Bars represent the mean colonies/field ± SEM of triplicate wells (p<0.05). FIG. 6C. Vector control and MDA/ParvB3 cells were cultured on collagen I and assayed for differences in proliferation using the MTT dye assay. Similar results were obtained comparing cells that were plated on tissue culture plastic. These results are representative of two independent determinations, with error bars denoting ±SEM of quadruplicate wells.

FIG. 7 shows ParvB inhibits matrigel invasion by MDA-MB-231 cells. FIG. 7A. Vector [symbol] and ParvB3 (▪) cells were assayed for adhesion to ECM. Cells were plated on collagen I for 1 hr at 37° C., and adherent cells visualized by crystal violet staining. Error bars indicate SEM of four experimental values (p<0.05). FIG. 7B. MDA/ParvB3 and vector control cells were plated on Matrigel, in the upper chamber of invasion plate wells, with bottom chambers containing medium with or without 100 ng/ml EGF. Results are presented as number of cells per microscope field (x10 objective) from five randomly selected fields. Data are expressed as the mean cells/field ± SEM of three chambers (p<0.05). Each experiment is representative of two independent determinations.

FIG. 8 shows ParvB3 suppresses EGF signaling and ILK kinase activity in breast carcinoma cells. FIG. 8A. MDA/ParvB3, MCF7/ParvB3 and vector control cells were serum-starved for 24 hours, and stimulated or not with EGF (100 ng/ml) for 10 minutes. Total cellular proteins (50 μg/lane) were resolved by 12% SDS-PAGE. Levels of phosphorylated GSK3 (GSK3-ser9), total GSK3 (GSK3), ILK, and GAPDH were determined by western blot. These results represent four independent determinations. FIG. 8B. Vector control and ParvB3 MDA-MB-231 cells were serum starved and stimulated with or without EGF. Cytoplasmic lysates (700μg) were analyzed in an ILK immune complex kinase assay using MBP as exogenous substrate.

FIG. 9 shows ParvB3 is an inhibitor of EGFR and ILK signaling. FIG. 9A. MDA/ParvB3 and vector control cells were infected with Ad-ILK at moi of 0, 1, 5, or 10 and immunoblotted for ILK expression after 48 hr. FIG. 9B. MDA/ParvB3 and vector control cells were infected with Ad-ILK for 24 hours. After infection, cells were serum-starved overnight and treated with or without EGF as indicated for 10 minutes. Total cellular proteins (50 μg/lane) were analyzed by immunoblotting with antibodies recognizing ILK, phosphorylated GSK3 (GSK3-ser9) and PKB (PKB-ser473), total GSK3 (GSK3) and PKB (PKB), and GAPDH. C. EGFR activation was confirmed by Western blotting with phospho-EGFR (Tyr1068) and EGFR antibodies. Each experiment represents at least two independent determinations.

FIG. 10 shows ParvB3 directly associates with ILK. FIG. 10A. EGY48 strain transformed with plasmids encoding ParvB3 cDNA, lacZ reporter, and the indicated ILK baits. LexILK^{WT} is kinase active, while LexILK^{E359K} is a kinase deficient variant, due to a point mutation in the catalytic domain. LexILK^{Δcat} is a truncated ILK (containing residues 1-276), lacking the catalytic domain. Both ILK^{E359K} and ILK^{Δcat} mutants showed very weak interactions, as compared to ILK^{WT}, indicated by the decreased intensity of blue colonies. FIG. 10B. Expression of LexA bait proteins in EGY48. Western blotting of transformed yeast lysates using a LexA monoclonal antibody indicated equivalent expression of ILK^{WT}, ILKE^{359K} and ILK^{Δcat} bait proteins. Results shown are representative of six colonies, isolated from each of three independent co-transformations of each ILK bait plasmid.

### DETAILED DESCRIPTION OF THE PREFERRED EMBODIMENTS

The present invention concerns the characterization of ParvB. The ParvB gene has now been characterized as a tumor suppressor gene involved in the regulation of breast cancer. The invention enables screening methods for the diagnosis, prognosis and prediction of breast cancer in subjects and also enables therapeutic methods for the treatment of breast cancer. In one aspect, ParvB and its expression products can now be used to inhibit tumor growth, notably breast cancer growth in vivo by various therapies such as but not limited to gene therapy.

With the knowledge of the function of ParvB, the ParvB nucleotide sequence, ParvB gene product, probes and antibodies raised to the gene product can be used in a variety of hybridization and immunological assays to screen for and detect the presence of ParvB, and ParvB expression relative to controls and the presence of any mutated gene or gene product in a suitable sample, such screening being for the purpose of diagnosis of breast cancer and for assessing progression and prognosis of breast cancer.

Breast tumors and breast cancer cell lines were analyzed for the expression β-parvin (ParvB), an adaptor protein that binds to the integrin linked kinase (ILK). Quantitative RT-PCR indicated ParvB mRNA was down-regulated, by at least 60%, in four of nine breast tumors, relative to patient-matched normal mammary gland tissue. ParvB protein levels were reduced by ≧90% in five of seven advanced tumors, relative to matched normal breast tissue. Conversely, ILK protein and kinase activity levels were elevated in these tumors, suggesting that downregulation of ParvB stimulates ILK signaling. RT-PCR and western blot analyses indicated very low levels of ParvB mRNA and protein in MDA-MB-231 and MCF7 breast cancer cells, facilitating functional studies of the effects of ParvB on ILK signaling. Expression of ParvB in MDA-MB-231 and MCF7 cells increased cell adhesion to collagen. ParvB inhibited ILK kinase activity, anchorage independent cell growth, and in vitro matrigel invasion by MDA-MB-231 cells. EGF-induced phosphorylation of two ILK targets, PKB (Ser473) and GSK3β (Ser9), was also inhibited by ParvB. These results indicated that ParvB inhibits ILK signaling downstream of receptor tyrosine kinases. Together these results suggest that loss of ParvB expression is a novel mechanism for upregulating ILK activity in tumors. As such ParvB can now be characterized as a tumor suppressor gene.

Tumor suppressor genes are typically thought of as genes whose expression is reduced or lost in cancer cells (Knudson, Proc. Natl. Acad. Sci. USA 19 (1993) 10914-10921). The lack of expression results from mutations in the genes encoding their proteins. Since these proteins are believed to suppress cell growth and thereby act as negative growth regulators, loss of their expression in tumor cells leads to the increased cell proliferation observed and contributes to malignant transformation. As negative growth regulators, tumor suppressor gene products are likely to have also normal functions critical to the development of differentiated tissues. In this respect, tumor suppressor genes may have an important role in the growth arrest necessary for the onset of cellular differentiation as growth regulation is a normal feature of development and differentiation. An overview of tumor suppressor genes is given by, e.g., Gutmann, D. H., Int. J. Dev. Biol. 39 (1995) 895-907.

Inactivation of tumor suppressor genes appears to be a predominant genetic event in the genesis and progression of many tumors. In normal cells, these genes are thought to be involved in the regulation of cell proliferation and differentiation (Fearon, E., and Vogelstein, B., Cell 61 (1990) 759-767). Inactivating mutations and deletions of tumor suppressor genes may therefore release normal growth constraints and may result in the development or progression of tumor cells. A genomic region that contains a putative tumor suppressor gene can be identified by frequent loss of heterozygosity (LOH) of the normal allele with the remaining allele being presumably non-functional in the tumor cells (Fearon, E., and Vogelstein, B., Cell 61 (1990) 759-767).

The present invention also relates to the correlation of ParvB expression and its association with breast cancer. The invention enables screening methods for the diagnosis of breast cancer and also enables therapeutic methods for the treatment of breast cancer.

With the knowledge of the ParvB gene sequence and the ParvB gene products (ParvB1-3), probes and antibodies raised to the gene product can be used in a variety of hybridization and immunological assays to screen for and detect the presence and levels of either a normal or mutated gene or gene product. Any such mutations that disrupt ILK-ParvB interaction may be used as predictive, diagnostic or prognostic for breast cancer.

Patient therapy through supplementation with the normal gene product by amplification, by genetic and recombinant techniques can now be achieved. Upregulation of the ParvB gene product by protein treatment together with wild-type supplementation is now also possible. Malignancies may be controlled by gene therapy in which recombinant ParvB is provided to be expressed in situ to deliver the normal gene product. The present invention also allows for the development of novel drugs to mimic the effect of the normal protein or for drugs used as antagonists of the protein.

### Definitions

The term “polynucleotide” denotes for example DNA, RNA or derivatized active DNA or RNA.

The term “hybridize under stringent conditions” means that two nucleic acid fragments are capable of hybridization to one another under standard hybridization conditions described in Sambrook et al., “Expression of cloned genes in E. Coli: in Molecular Cloning: A laboratory manual (1999) Cold Spring Harbor Laboratory Press, New York, USA.

A functional fragment of a tumor suppressor gene product, means any proteineous molecule that retains its tumor suppression activity. A variant of a tumor suppressor gene product according to the invention is a gene product with at least 60% identity, preferably 70% identity, more preferably 80% identity, most preferably 90% sequence identity to said tumor suppressor gene product, as measured by a BLASTP or TBLASTN search (Altschul et al., 1997), and retaining its tumor suppression activity. An isolated nucleic acid encoding a tumor suppressor gene product means that said nucleic acid comprises partly or totally the coding sequence of said tumor suppressor gene. The definition covers, but is not limited to genomic DNA and messenger RNA. It does however not implicate that said genomic DNA is transcribed and translated into said tumor gene product. A functional fragment of a nucleic acid for use in diagnosis of and/or prediction of the likelihood of means any fragment that can be used as specific probe in hybridization or as primer in PCR reactions. A functional fragment of a nucleic acid for use in the treatment of cancer means any fragment that can be transcribed and/or translated into a functional tumor suppressor. A functional fragment of a tumor suppressor gene product for the manufacture of a medicament to treat cancer is any fragment of said tumor suppressor gene product that retains its tumor suppression function. A functional fragment of a tumor suppressor gene product in the production of antibodies is an immunogenic fragment that comprises at least one epitope and can be used for the production of antibodies against said tumor suppressor gene product. A functional fragment of a tumor suppressor gene product in the isolation of an interacting compound is any fragment that can be used in an interaction screening assay, such as, but not limited to, a yeast two-hybrid assay (for example CytoTrap™ from Strategne), a phage display assay, co-immunoprecipitation, a DNase protection assay, an electrophoretic mobility shift assay, or mass spectrometric analyses.

The terms protein and polypeptide as used in this application are interchangeable. Polypeptide refers to a polymer of amino acids and does not refer to a specific length of the molecule. This term also includes post-translational modifications of the polypeptide, such as glycosylation, phosphorylation and acetylation. Compound as used here means any chemical or biological compound, including simple or complex organic or inorganic molecules, peptides, peptido-mimetics, proteins, antibodies, carbohydrates, nucleic acids or derivatives thereof. Interacting compound with a protein means any compound that can bind, covalently or not, with said protein in a specific way.

ParvB proteins, fragments of the proteins and fusion proteins may be isolated and purified by techniques well known to those skilled in the art. The ParvB protein may be purified from tissues (e.g. mammary tissue) in which there is a high level of expression of the protein or it may be made by recombinant techniques. Isolated proteins, or fragments thereof can be used for the generation of antibodies, in the identification of proteins which may bind to ParvB or for diagnostic or therapeutic methods and assays. Full length proteins and fragments of at least about 4 amino acids may be isolated and purified for various applications The protein may be isolated from tissue by extraction and solubilized using a detergent. Purification can be achieved using protein purification procedures such as chromatography methods (gel-filtration, ion-exchange and immunoaffinity), by high-performance liquid chromatography (RP-HPLC, ion-exchange HPLC, size-exclusion HPLC, high-performance chromatofocusing and hydrophobic interaction chromatography) or by precipitation (immunoprecipitation or immunoaffinity SDS-Page and Page). Polyacrylamide gel electrophoresis can also be used to isolate the ParvB protein based on its molecular weight, charge properties and hydrophobicity. Similar procedures may be used to purify the protein from recombinant expression system.

For protein expression, eukaryotic or prokaryotic expression systems may be generated in which the ParvB gene sequence, cDNA (for example as provided in Accession No. Q9HBI1, Q5TGJ5 Q86X93 Q9NSP7 Q9UGT3 Q9Y368 Q9Y3L6 Q9Y3L7; Olski T. M., Noegel A. A., Korenbaum E.;“Parvin, a 42 kDa focal adhesion protein, related to the alpha-actinin superfamily.”; J. Cell Sci. 114:525-538(2001); Yamaji S., Suzuki A., Sugiyama Y., Koide Y., Yoshida M., Kanamori H., Mohri H., Ohno S., Ishigatsubo Y.; “A novel integrin-linked kinase-binding protein, affixin, is involved in the early stage of cell-substrate interaction.”; J. Cell Biol. 153:1251-1264(2001); DOI=10.1101/gr.10.5.703; Lai C.-H., Chou C.-Y., Ch′ang L.-Y., Liu C.-S., Lin W.-C.; “Identification of novel human genes evolutionarily conserved in Caenorhabditis elegans by comparative proteomics.”; Genome Res. 10:703-713(2000)) or genomic (Dunham I., Hunt A. R., Collins J. E., Bruskiewich R., Beare D. M., Clamp M., Smink L. J., Ainscough R., Almeida J. P., Babbage A. K., Bagguley C., Bailey J., Barlow K. F., Bates K. N., Beasley O. P., Bird C. P., Blakey S. E., Bridgeman A. M., Buck D., Wright H.; “The DNA sequence of human chromosome 22.”; Nature 402:489-495(1999)), is introduced into a plasmid or other expression vector which is then introduced into living cells. Constructs in which the ParvB cDNA sequence containing the entire open reading frame inserted in the correct orientation into an expression plasmid may be used for protein expression (see Example Section). Alternatively, portions of the ParvB sequences may be inserted. Typical expression vectors contain promoters that direct the synthesis of large amounts of mRNA corresponding to the gene. They may also include sequences allowing for their autonomous replication within the host organism, sequences that encode genetic traits that allow cells containing the vectors to be selected, and sequences that increase the efficiency with which the mRNA is translated. Stable long-term vectors may be maintained as freely replicating entities by using regulatory elements of viruses. Cell lines may also be produced which have integrated the vector into the genomic DNA and in this manner the gene product is produced on a continuous basis.

Expression of foreign sequences in bacteria such as E. coli require the insertion of the sequence into an expression vector, usually a plasmid which contains several elements such as sequences encoding a selectable marker that assures maintenance of the vector in the cell, a controllable transcriptional promoter which upon induction can produce large amounts of mRNA from the cloned gene, translational control sequences and a polylinker to simplify insertion of the gene in the correct orientation within the vector. A relatively simple E. coli expression system utilizes the lac promoter and a neighbouring lacZ gene which is cut out of the expression vector with restriction enzymes and replaced by the ParvB gene sequence.

In vitro expression of proteins encoded by cloned DNA may also be done using the T7 late-promoter expression system. Plasmid vectors containing late promoters and the corresponding RNA polymerases from related bacteriophages such as T3, T5 and SP6 may also be used for in vitro production of proteins from cloned DNA. E. coli can also be used for expression by infection with M13 Phage mGPI-2. E. coli vectors can also be used with phage lambda regulatory sequences, with fusion protein vectors, with maltose-binding protein fusions, and with glutathione-S-transferase fusion proteins.

Eukaryotic expression systems permit appropriate post-translational modifications of expressed proteins. This allows for studies of the ParvB gene and gene product including determination of proper expression and post-translational modifications for biological activity, identifying regulatory elements in the 5′ region of the gene and their role in tissue regulation of protein expression. It also permits the production of large amounts of protein for isolation and purification, the use of cells expressing ParvB as a functional assay system for antibodies generated against the protein, the testing of the effectiveness of pharmacological agents or to increase or decrease the activity of ParvB, and the study of the function of the normal complete protein, specific portions of the protein, or of naturally occurring polymorphisms and artificially produced mutated proteins.

In order to produce mutated/altered or polymorphic proteins, the ParvB DNA sequence can be altered using procedures such as restriction enzyme digestion, DNA polymerase fill-in, exonuclease deletion, terminal deoxynucleotide transferase extension, ligation of synthetic or cloned DNA sequences and site-directed sequence alteration using specific oligonucleotides together with PCR. Once an appropriate expression vector containing the ParvB gene is constructed, it is introduced into an appropriate E. coli strain by transformation techniques including calcium phosphate transfection, DEAE-dextran transfection, electroporation, microinjection, protoplast fusion and liposome-mediated transfection.

The host cell to be transfected with the vector of this invention may be selected from the group consisting of E.coli, Pseudomonas, Bacillus Subtilis, or other bacilli, other bacteria, yeast, fungi, insect (using baculoviral vectors for expression), mouse or other animal or human tissue cells. Mammalian cells can also be used to express the PARVB protein using a vaccinia virus expression system. Prokaryotic and eukaryotic expression systems allow various important functional domains of the protein to be recovered as fusion proteins and used for binding, structural and functional studies and also for the generation of appropriate antibodies.

In order to express and purify the protein as a fusion protein, the ParvB cDNA sequence is inserted into a vector which contains a nucleotide sequence encoding another peptide (eg. GST--glutathionine succinyl transferase). The fusion protein is expressed and recovered from prokaryotic (eg. bacterial or baculovirus) or eukaryotic cells. The fusion protein can then be purified by affinity chromatography based upon the fusion vector sequence and the PARVB protein obtained by enzymatic cleavage of the fusion protein. Fusion proteins are particularly advantageous because they provide a system for ensuring a good expression of the protein without making any alternations to the 5′ end of the coding sequence or immediately preceding the start codon. In the fusion approach, a cloned gene is introduced into an expression vector 3′ or 5′ to add a C-terminal tag to a carrier sequence coding for the amino terminus of a highly expressed protein. The carrier sequence provides the necessary signals for good expression and the expressed fusion protein contains a N terminal region encoded by the carrier. Purified protein can also be used in further biochemical analyses to establish secondary and tertiary structure. The preparation of substantially purified ParvB protein or fragments thereof allows for the determination of the protein tertiary structure. by x-ray crystallography of crystal of ParvB protein or by NMR. Determination of structure may aid in the design of pharmaceuticals to interact with the protein, alter protein charge configuration or charge interaction with other proteins, or to alter its function in the cell.

The knowledge of the amino acid and nucleotide sequence of ParvB allows for the production of antibodies which selectively bind the ParvB protein (and any of the protein isoforms ParvB1, ParvB2 or ParvB3) or fragments thereof. Antibodies can also be made to selectively bind and/or distinguish altered forms of normal protein. In order to prepare polyclonal antibodies, fusion proteins containing defined portions or all of the ParvB protein can be synthesized in bacteria by expression of corresponding DNA sequences in a suitable cloning vehicle. Fusion proteins are commonly used as a source of antigen for producing antibodies. Alternatively protein may be isolated and purified from PARVB expressing cultures and used as a source of antigen. It is understood that the entire protein or fragments thereof can be used as a source of antigen to produce antibodies (see example section).

The purified ParvB protein (or isoforms thereof) is purified, coupled to a carrier protein and mixed with adjuvant such as but not limited to Freund's adjuvant (to help stimulate the antigenic response by the animal) and injected into rabbits or other appropriate laboratory animals. Following booster injections at weekly intervals, the rabbits or other laboratory animals are then bled and the sera isolated. The sera can be used directly or purified prior to use by various methods including affinity chromatography employing Protein A-Sepharose, Antigen Sepharose or Anti-mouse-Ig-Sepharose, to give polyclonal antibodies. Alternatively, synthetic peptides can be made corresponding to the antigenic portions of the protein, and used to inoculate the animals. The most common practice is to choose a 10 to 15 amino acid residue peptide corresponding to the carboxyl or amino terminal sequence of a protein antigen, and to chemically cross-link it to a carrier molecule such as keyhole limpet hemocyanin or BSA. However, if an internal sequence peptide is desired, selection of the peptide is based on the use of algorithms that predict potential antigenic sites. These predictive methods are, in turn, based on predictions of hydrophilicity (Kyte and Doolittle 1982, Hopp and Woods 1983) or secondary structure (Chou and Fasman 1978). The objective is to choose a region of the protein that is either surface exposed, such as a hydrophilic region, or is conformationally flexible relative to the rest of the structure, such as a loop region or a region predicted to form a .beta.-turn. The selection process is also limited by constraints imposed by the chemistry of the coupling procedures used to attach peptide to carrier protein. Carboxyl-terminal peptides are frequently chosen because these are often more mobile than the rest of the molecule and the peptide can be coupled to a carrier in a straightforward manner using glutaraldehyde. The amino-terminal peptide has the disadvantage that it may be modified post-translationally by acetylation or by the removal of a leader sequence. A comparison of the protein amino acid sequence between species can yield important information. Those regions with sequence differences between species are likely to be immunogenic. Synthetic peptides can also be synthesized as immunogens as long as they mimic the native antigen as closely as possible.

It is understood by those skilled in the art that monoclonal ParvB antibodies may also be produced. ParvB protein isolated from tissues, or from cells recombinantly expressing the protein, is injected in Freund's adjuvant into mice. Mice are injected 9 times over a three week period, after which their spleens are removed and re-suspended in phosphate buffered saline (PBS). The spleen cells serve as a source of lymphocytes, some of which are producing antibody of a selected specificity. These are then fused with a permanently growing myeloma partner cell, and the products of the fusion are plated into a number of tissue culture wells in the presence of a selective agent such as HAT. The wells are then screened by ELISA to identify those containing cells making binding antibody. These are then plated and after a period of growth, these wells are again screened to identify antibody-producing cells. Several cloning procedures are carried out until over 90% of the wells contain single clones which are positive for production of the desired antibody. From this procedure, a stable line of clones which produce the antibody is established. The monoclonal antibody can then be purified by affinity chromatography using Protein A Sepharose, ion-exchange chromatography, as well as variations and combinations of these techniques. Truncated versions of monoclonal antibodies may also be produced by recombinant techniques in which plasmids are generated which express the desired monoclonal antibody fragment(s) in a suitable host. Antibodies specific for mutagenised epitopes can also be generated. These antibodies are especially useful in cell culture assays to screen for malignant cells at different stages of malignant development. Such antibodies are also useful for screening malignant cells which have been treated with pharmaceutical agents in order to evaluate the therapeutic potential of the pharmaceutical agent. ParvB antibodies are also useful for detecting both normal and mutant proteins in cell culture, and transfected cell cultures expressing normal or mutant PARVB protein as well as for western blot analysis on protein extracts of such cells.

Antibodies are also useful in various immunoassays for detecting and quantitating relative amounts of ParvB (ParvB1-3) protein and fragments thereof. Enzyme-linked immunosorbant assays (ELISA) may be used to detect ParvB as well as antibodies generated against these proteins. Commonly used ELISA systems are indirect ELISA to detect specific antibodies, direct competitive ELISA to detect soluble antigens, antibody-sandwich ELISA to detect soluble antigens and double antibody-sandwich ELISA to detect specific antibodies.

Antibodies to ParvB may also be used for coupling to compounds such as radionuclides or fluorescent compounds, or to liposomes for diagnostic imaging and therapy, in order to target compounds to a specific tissue location. This is especially valuable for the specific targeting of malignant tissues with anti-cancer drugs, which would be detrimental to normal cells and tissues. For a review of methods for preparation of antibodies, see Antibody Engineering: A Practical Guide, Barreback, ed., W. H. Freeman & Company, N.Y. (1992) or Antibody Engineering, 2nd Ed., Barreback, ed., Oxford University Press, Oxford (1995).

The ParvB gene and gene product, as well as the ParvB-derived probes, primers and antibodies, disclosed or otherwise enabled herein, are useful in the screening for carriers of alleles associated with breast cancer, for the diagnosis of victims of cancer, and for the monitoring of the prognosis of those diagnosed with breast cancer. Individuals at risk for developing breast cancer possibly those being at risk due to a family pedigree, or individuals not previously known to be at risk, may be routinely screened using probes to detect the presence of a ParvB gene or ParvB protein by a variety of techniques. Diagnosis of inherited cases of these diseases can be accomplished by methods based upon the nucleic acid (including genomic and mRNA/cDNA sequences), proteins, and/or antibodies disclosed and enabled herein, including functional assays designed to detect failure or augmentation of the normal ParvB activity or the presence of specific new activities conferred by a mutant ParvB. The methods and products are based upon the human ParvB nucleic acids, protein or antibodies, as disclosed or otherwise enabled herein.

As will be appreciated by one of ordinary skill in the art, the choice of diagnostic methods of the present invention will be influenced by the nature of the available biological samples to be tested and the nature of the information required. ParvB is expressed in breast mammary tissue and thus breast tissue may be used as a source of biological sample as well as any tissue representing metastatic sites for breast cancer, such as but not limited to bone and lymph nodes.

A variety of approaches are available for a diagnostic assay based upon the ParvB protein. For example, diagnosis can be achieved by monitoring differences in the electrophoretic mobility of normal and any mutant proteins. Such an approach will be particularly useful in identifying mutants in which charge substitutions are present, or in which insertions, deletions or substitutions have resulted in a significant change in the electrophoretic migration of the resultant protein. Alternatively, diagnosis may be based upon differences in the proteolytic cleavage patterns of normal and any mutant proteins, differences in molar ratios of the various amino acid residues, or by functional assays demonstrating altered function of the gene products. In embodiments, protein-based diagnostics will employ differences in the ability of antibodies to bind to normal and any mutant ParvB proteins. Such diagnostic tests may employ antibodies which bind to the normal proteins but not to mutant proteins, or vice versa. In particular, an assay in which a plurality of monoclonal antibodies, each capable of binding to a mutant epitope, may be employed. The levels of anti-mutant antibody binding in a sample obtained from a test subject (visualized by, for example, by radiolabelling, ELISA or chemiluminescence) may be compared to the levels of binding to a control sample. Alternatively, antibodies which bind to normal but not mutant ParvB may be employed, and decreases in the level of antibody binding may be used to distinguish homozygous normal individuals from mutant heterozygotes or homozygotes. Such antibody diagnostics may be used for in situ immunohistochemistry using biopsy samples of tissues obtained antemortem or postmortem.

Diagnostic assays based upon nucleic acids from a sample may employ mRNA, cDNA or genomic DNA. Whether mRNA, cDNA or genomic DNA is assayed, standard methods well known in the art may be used to detect the presence of a particular sequence either in situ or in vitro (see, e.g., Sambrook et al., (1989) Molecular Cloning: A Laboratory Manual, 2nd ed., Cold Spring Harbor Press, Cold Spring Harbor, N.Y.). As a general matter, however, any tissue with nucleated cells may be examined. Genomic DNA used for the diagnosis may be obtained from body cells, such as those present in a tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, direct nucleotide sequencing, hybridization using specific oligonucleotides, restriction enzyme digest and mapping, PCR mapping, RNase protection, chemical mismatch cleavage, ligase-mediated detection, and various other methods may be employed. Oligonucleotides specific to particular ParvB sequences can be chemically synthesized and labeled radioactively or non-radioactively (e.g., biotin tags, ethidium bromide), and hybridized to individual samples immobilized on membranes or other solid-supports (e.g., by dot-blot or transfer from gels after electrophoresis), or in solution. The presence, absence or comparative reduction of the target sequences may then be visualized using methods such as autoradiography, fluorometry, or colorimetry. These procedures can be automated using redundant, short oligonucleotides of known sequence fixed in high density to silicon chips.

Whether for hybridization, RNase protection, ligase-mediated detection, PCR amplification or any other standards methods described herein and well known in the art, a variety of subsequences of the ParvB sequences (ParvB1-3) disclosed or otherwise enabled herein will be useful as probes and/or primers. These sequences or subsequences will include both normal ParvB sequences and any possible deleterious mutant sequences. In general, useful sequences will include at least 8-9, more preferably 10-50, and most preferably 18-24 consecutive nucleotides from the ParvB introns, exons or intron/exon boundaries. Depending upon the target sequence, the specificity required, and future technological developments, shorter sequences may also have utility. Therefore, any ParvB derived sequence which is employed to isolate, clone, amplify, identify or otherwise manipulate a ParvB sequence may be regarded as an appropriate-probe or primer. Particularly contemplated as useful will be sequences including nucleotide positions from the ParvB genes from sequences encoding the CH1 and CH2 domains.

Therapies may be designed to circumvent or overcome a ParvB gene defect or inadequate ParvB gene expression, and thus moderate and possibly prevent malignancy. The ParvB gene has been found to be down regulated or not expressed in breast cancer. In considering various therapies, however, it is understood that such therapies may be targeted at other malignant tissues in which ParvB is demonstrated to have a tumor suppressor function.

Treatment or prevention of breast cancer can be accomplished by replacing a mutant ParvB protein with normal protein, by modulating the function of a mutant protein, or by delivering normal ParvB protein to the appropriate cells. It is also be possible to modify the pathophysiologic pathway (signal transduction pathway) in which the protein participates in order to correct the physiological defect such as by affecting ILK function.

To replace a mutant protein with normal protein, or to add protein to cells which no longer express ParvB, or express inadequate amounts of ParvB it is necessary to obtain large amounts of pure ParvB protein from cultured cell systems which can express the protein. Delivery of the protein to the affected cells and tissues can then be accomplished using appropriate packaging or administration systems. Alternatively, small molecule analogs may be used and administered to act as ParvB agonists or antagonists and in this manner produce a desired physiological effect. In order to screen for analogues, one can design functional screens based on the sequence of ParvB. One can also fuse ParvB to heterologous DNA binding proteins to design screens for agonists. Since ParvB functions by interacting with other proteins such as ILK, yeast screens can be used for small molecules that may interact by promoting or disrupting ParvB binding with other proteins.

Based on the biochemical analyses of ParvB protein structure-function, one can design drugs to mimic the effects of ParvB on target proteins. Recombinant ParvB expressed as a fusion protein can be utilized to identify small peptides that bind to ParvB such as by using a phage display approach. An alternate but related approach uses the yeast two hybrid system to identify further binding partners for ParvB. ParvB or fragments are expressed in yeast as a fusion to a DNA binding domain. This fusion protein is capable of binding to target promoter elements in genes that have been engineered into the yeast. These promoters drive expression of specific reporter genes (Typically the auxotrophic marker HIS3 and the enzyme β-galactosidase). A library of cDNAs can then be constructed from any tissue or cell line and fused to a transcriptional activation domain. Transcription of HIS3 and β-galactosidase depends on association of the ParvB fusion protein (which contains the DNA binding domain) and the target protein (which carries the activation domain). Yeast survival on specific growth media lacking histidine requires this interaction. This approach allows for the identification of specific proteins that interact with ParvB. The approach has also been adapted to identify small peptides. ParvB, or its fragments, are fused with the DNA binding domain and are screened with a library of random peptides or peptides which are constrained at specific positions linked to a transcriptional activation domain. Interaction is detected by growth of yeast containing the interacting peptides on media lacking histidine and by detection of β-galactosidase activity using standard techniques.

The identification of proteins or small peptides that interact with ParvB can provide the basis for the design of small peptide antagonists or agonists of ParvB function. Further, the structure of these peptides determined by standard techniques such as protein NMR or X-ray crystallography can provide the structural basis for the design of small molecule drugs.

Gene therapy is another potential therapeutic approach in which normal copies of the ParvB gene are introduced into selected tissues such as mammary tissue to successfully code for normal protein in affected cell types. It is to be understood that gene therapy techniques may only begin once a malignant genotype/phenotype has been identified. The gene must be delivered to affected cells in a form in which it can be taken up and can code for sufficient protein to provide effective function. Transducing retroviral vectors can be used for somatic cell gene therapy especially because of their high efficiency of infection and stable integration and expression. The targeted cells must be able to divide and the expression level of normal protein should be high. The full length ParvB gene, or portions thereof, can be cloned into a retroviral vector and driven from its endogenous promoter or from the retroviral long terminal repeat or from a promoter specific for the target cell type of interest. Other viral vectors which can be used include adeno-associated virus, vaccinia virus, bovine papilloma virus, or a herpes virus such as Epstein-Barr virus. Gene therapy utilizing the ParvB nucleotide sequence may be used alone or in combination with therapeutic agents to control the growth of tumor cells.

Gene transfer could also be achieved using non-viral methods of infection in vitro. These methods would include calcium phosphate, DEAE dextran, electroporation, and protoplast fusion. Liposomes may also be potentially beneficial for delivery of DNA into a cell. Transplantation of normal genes into the affected cells of a patient with a malignancy can also be useful therapy, especially if the malignancy is diffuse and cannot be excised or if the tissue affected has been substantially transformed into a malignant phenotype and due to its function in the body, cannot be surgically removed. In this procedure, normal ParvB is transferred into a cultivatable cell type, either exogenously or endogenously to a patient. These cells are then injected serotologically into the targeted tissue(s).

Cell lines may be cultured which express ParvB, to which a test compound/agent is added to the culture medium. After a period of incubation, the expression of ParvB mRNA and resultant protein product can be quantified to determine any changes in expression as a result of the test compound/agent. Changes identified in this context are defined as an increase or decrease of at least 5% ParvB expression and protein production. In aspects, the levels of increase or decrease is greater than 10% or more. Cell lines transfected with constructs expressing mutant or normal ParvB can also be used to test the function of compounds/agents developed to modify the protein function. For example, compounds may be identified that encourage the interaction of ParvB with ILK. Alternatively, known breast cancer cell lines may be used in order to test compounds which can upregulate/increase ParvB expression and therefore increase ParvB protein amounts. With respect to normal ParvB, the effect of protein drugs/agents which interact with the protein's normal function could be studied in order to more precisely define the intracellular role of the protein. Cells useful for the assays of the present invention include any eukaryotic cells in which ParvB is expressed. In aspects, such cells are mammary tissue cells. It is with the elucidation of the exact function of the protein and the components of the signalling pathway that it is involved which will allow for the development of novel drugs to restore normal function of the protein. All testing for novel drug development is well suited to defined cell culture systems which can be manipulated to express normal or mutated ParvB and study the resultant signaling and gene transcription. Animal models are also important for testing novel drugs. Antibodies generated to recognize mutant ParvB and bind to novel drugs can be used to specifically target malignant tissues expressing any mutant ParvB. In this manner only malignant cells can be targeted with potentially lethal pharmaceutical agents and not normal surrounding tissues.

The ParvB nucleotide sequence and portions/fragments thereof, polypeptide sequence and portions/fragments thereof, including functional analogues thereof may be incorporated into a pharmaceutical composition for the treatment of breast cancer. Administration of the various ParvB composition embodiments is accomplished by methods well known to those skilled in the art. Such administration can be either alone or in acceptable pharmaceutical mediums. A pharmaceutically acceptable carrier can contain a physiologically acceptable compound that acts, for example, to stabilize the composition or to increase or decrease the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of a pharmaceutically acceptable carrier, including a physiologically acceptable compound, depends, for example, on the route of administration of the polypeptide and on the particular physio-chemical characteristics of the specific polypeptide. For example, a physiologically acceptable compound such as aluminum monosterate or gelatin is particularly useful as a delaying agent, which prolongs the rate of absorption of a pharmaceutical composition administered to a subject. Further examples of carriers, stabilizers or adjuvants can be found in Martin, Remington's Pharm. Sci., 15th Ed. (Mack Publ. Co., Easton, 1975), incorporated herein by reference. The pharmaceutical composition also can be incorporated, if desired, into liposomes, microspheres or other polymer matrices (Gregoriadis, Liposome Technology, Vol. 1 (CRC Press, Boca Raton, Fla. 1984), which is incorporated herein by reference). Liposomes, for example, which consist of phospholipids or other lipids, are nontoxic, physiologically acceptable and metabolizable carriers that are relatively simple to make and administer. Vectors containing a ParvB coding sequence can be administered as pharmaceutical compositions which include the vectors described herein in combination with one or more of the above pharmaceutically acceptable carriers. The compositions can then be administered therapeutically or prophylactically. Methods of administering a pharmaceutical containing the vector of this invention, are well known in the art and include but are not limited to, administration orally, intra-tumorally, intravenously, intramuscularly or intraperitoneal. Administration can be effected continuously or intermittently and will vary with the subject and the condition to be treated, e.g., as is the case with other therapeutic compositions (Landmann et al. (1992); Aulitzky et al. (1991); Lantz et al. (1990); Supersaxo et al. (1988); Demetri et al. (1989); and LeMaistre et al. (1991)).

The present invention also embodies the provision of diagnostic kits for the diagnosis of breast cancer. Such kits may include probes, primers directed to the ParvB sequence or portion thereof or functional analogue thereof of the ParvB nucleic acid sequence. Kits may further include reagents and/or instructions for use. Therapeutic kits may contain vectors that can express ParvB polypeptide.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Construction of ParvB Expression Olasmids

For cloning into pcDNA3.1, a full length ParvB3 cDNA was isolated from a human adult heart library (Clontech) by screening with a ParvB3 RT-PCR derived probe. The following primers were then used to amplify cDNA inserts, using high-fidelity Pfu DNA polymerase (MBI Fermentas):

| | |
|---|---|
| 5′-GGCCGGTACCATGCACCATGTGTTTAAA | (forward) |
| 3′-GGCCGATATCCTCCACGTTCTTGTACTT | (reverse), |

The PCR product was digested with KpnI and EcoRV restriction enzymes, gel-purified, and ligated into KpnI/EcoRV digested pcDNA3.1 vector. Following transformation of E. coli DH5α, colonies were screened for the presence of inserts by KpnI and EcoRV restriction digestions. Positive clones were sequenced to verify cloning in-frame with the myc-His epitope tag.

For expression of GFP-ParvB fusion proteins, full-length PARVB3 (residues 1-397) and two truncated cDNAs, CH1(1-142) and CH2(157-397) corresponding to residues 1-142 and 157-397 respectively, were generated. The pcDNA-ParvB3 plasmid was used as template for PCR cloning into the pEGFP-C3 expression vector (Clontech). The PCR products were generated with high-fidelity Pfu DNA polymerase (Stratagene) using the following primers:

ParvB3:

5′- GGCCGAGCTCATGCACCATGTGTTTAAAG -3′
5′- GGCCCCGCGGTCACTCCACGTTCTTGTAC -3′

CH1(1-142):

5′- GGCCGAGCTCATGCACCATGTGTTTAAAG -3′
5′- GGCCCCGCGGCGTTACATGCTCAGGA -3′

CH2(157-397):

5′- GGCCGAGCTCTCCAGCCACATCTCGGAG -3′
5′- GGCCCCGCGGTCACTCCACGTTCTTGTAC -3′

PCR products were digested with SacI and SacII restriction enzymes (MBI), gel-purified, and ligated into SacI/SacII digested pEGFP-C3 vector. Following transformation, colonies were screened for the presence of inserts by SacI and SacII restriction digestions. Positive clones were sequenced to verify cloning in-frame with the EGFP epitope tag.

### Construction of ILK Adenovirus

Full length ILK cDNA (Hannigan et al., 1996) was subcloned into a shuttle vector, pAdTrack-CMV (AdEasy System, Stratagene). The plasmid was linearized by restriction digestion with PmeI and cotransformed into E. coli BJ5183 cells with pAdEasy-1. Recombinants were selected for kanamycin resistance and recombination confirmed by restriction analysis. The linearized recombinant plasmid was transfected into an adenovirus packaging cell line, HEK293, using Lipofectamine (Invitrogen) in T-25 flasks (2×10⁶ cells/flask) according to manufacturer's instructions. Transfections and viral productions were monitored by GFP expression from the pAdTrack vector. For viral purification cells were harvested and subjected to four freeze/thaw cycles in dry ice/methanol and centrifuged to remove cell debris. Supernatant containing virus (8 mis) was combined with 4.4g CsCI and centrifuged at 32,000 rpm, 10° C., for 24 hours. The virus fraction was collected and titred on HEK293 cells.

### Production of Polyclonal ParvB Antibodies

A polyclonal β-parvin (PARVB) antiserum was generated, by immunizing New Zealand white rabbits with sarkosyl-solubilized GST-ParvB3 fusion protein, comprising amino terminal residues 1-142 of ParvB3. Amino acid numbering is in reference to the ParvB3 translation product, which we initially deposited to GenBank as CLINT (accession AAL08219). For immunodepletion of GST antibodies, crude serum from the immunized rabbits was incubated with recombinant GST bound to Glutathione Sepharose 4B (Pharmacia Biotech). The depleted serum was then incubated with GST-ParvB3 recombinant protein immobilized on Glutathione Sepharose 4B. ParvB antibodies were subsequently eluted from the washed column with 100 mM glycine (pH 2.8) and immediately neutralized in 1 M Tris-HCl (pH 8.0) as described (Youssoufian, 1998).

### Cell Culture and Derivation of Stable Cell Lines

Human breast cancer cell lines were maintained in Dulbecco's α-minimal essential medium (α-MEM), supplemented with 10% fetal calf serum (FCS) (Invitrogen) and antibiotic/antimycotic (100x) (Invitrogen). Wild-type β-parvin pcDNA3.1/Myc-His B clone or the vector alone was introduced into MDA-MB-231 and MCF7 cells using Fugene 6 Transfection Reagent (Roche). Two days post-transfection cells were replated and grown in α-MEM containing 10% FCS and 1.4 mg/ml G418 (Invitrogen). After 21 days of selection, neomycin-resistant ParvB3 and empty vector cells were expanded.

### Cell Adhesion and Matrigel Invasion Assays

Cell adhesion assays were performed as previously described (Hannigan et al., 1996). Briefly, cells (5×10⁵) were resuspended in DMEM supplemented with 1x Hepes (Invitrogen) and seeded in quadruplicate wells in a 96-well plate, and incubated at 37° C. in a humidified CO₂ cabinet for 1 hour. Cells were washed with PBS once, fixed in 5% glutaraldehyde (Sigma) for 20 minutes at room temperature, and stained with 0.1% crystal violet solution (Sigma) at room temperature for 1 hour. Cell adhesion was quantified by measuring the absorbance at optical density 570nm using a Spectra Max 250 ELISA plate reader.

In vitro cell invasion was assayed in BD BioCoat matrigel invasion chambers (BD Biosciences, 24 wells, 8 micron pore size). The top chamber was seeded with 5×10⁴ cells in DMEM. The bottom chamber was filled with DMEM supplemented with EGF (100 ng/ml) as a chemoattractant. Chambers were incubated for 20 hours in a humidified tissue culture incubator, 37° C., 5% CO₂ atmosphere. Noninvasive cells were removed from the upper surface of the membrane with a cotton swab, and cells on the lower surface of the membrane were fixed and stained with Diff-Quick and mounted on glass slides. Five random fields/well (x 10 objective) were counted for quantitation of cell invasion. Triplicate wells were counted for each assay. Data were analyzed using Prism v. 3.0 statistical software (GraphPad, Inc.).

### Analysis of Cell Growth and Cell Cycling

Adherent cell growth was assessed by MTT dye conversion at 570 nm according to manufacturers recommendations (Roche Applied Science). Briefly, ParvB3 or vector control cells were seeded in 96-well plates (2×10³ cells/well) and incubated in either 0.50% or 10% FBS-containing medium for 96 hours. After the incubation period, MTT labeling reagent was added to each well. Following a 4 hour incubation at 37° C., formazan salt crystals were solubilized overnight in a humidified atmosphere. The solubilized formazan product was quantified spectrophotometrically using an ELISA reader (OD 570 nm).

For analysis of anchorage independent growth, cells (5-10×10⁴) stably transfected with vector or myc-tagged ParvB3 were suspended in soft (0.3%) Bacto agar (Difco) in α-MEM with 10% FBS and placed onto a solidified layer of 0.5% agar, in triplicate wells of a 6-well plate. Colonies were allowed to develop for 10 days. Average colony numbers were determined by counting colonies greater than 5 cells in five random fields in triplicate wells at 10x magnification.

### Analysis of ParvB mRNA and Protein Expression

Snap-frozen human breast cancer and adjacent normal mammary gland specimens were obtained from the Cooperative Human Tissue Network (CHTN) at the Hospital of the University of Pennsylvania in accordance with Institutional Review Board (IRB) standards and guidelines. Total RNA was prepared from human breast tissues using the RNeasy Lipid Tissue Midi Kit (Qiagen GmbH, Hilden, Germany) according to the manufacturer's instructions. Total RNA was prepared from breast cell lines using Trizol reagent (Invitrogen) according to the manufacturer's instructions. Single-stranded cDNA was synthesized from 5 μg total RNA using the Superscript™ First Strand Synthesis System for RT-PCR (Invitrogen) and resuspended in DEPC-treated water (100 μl total volume). PARVB PCR products were generated by touchdown PCR amplification of 5 μl of cDNA template (in a total volume of 50 μl) using MasterTaq polymerase (Eppendorf, Westbury, N.Y.) and a forward oligonucleotide primer in exon 10 (PARVB1Fn, 5′ TTTGGAGGTGACGGAACTGGA 3′) paired with a reverse primer in exon 13 (PARVB1Rn, 5′ TGAAGGCCTGTGATCGCTAAC 3′). Internal control PCR reactions for human β2-microglobulin were performed using a forward primer in exon 1 (βm2E1sl, 5′ AGATGTCTCGCTCCGTGGCCT 3′) paired with a reverse primer in exon 2 (βm2E2asl, 5′ CCCACTTAACTATCTTGGGCTGT 3′). PCR products were ligated into the pCRII vector using the TA cloning system (Invitrogen) followed by transformation of XL-1 Blue bacterial cells. White colonies were screened for the presence of PARVB cDNA insert by PCR amplification. Plasmid DNA was prepared using the QIAprep Spin Miniprep Kit (Qiagen) and the inserts sequenced.

Quantitative real-time PCR (Q-PCR) reactions were performed using SYBR green reagent (Applied Biosystems) in 25 μl total volume and an ABI PRISM® 7000 Sequence Detection System (Applied Biosystems) according to the manufacturer's instructions. PARVB primers were designed to lie in different exons. For PARVB, 1 μl of cDNA template was used with forward primer hPARVBrealF (5′CATCCGCCTTCCTGAGCAT 3′) paired with reverse primer hPARVBrealR (5′AGCAGGCCTTCCCGTTTC 3′) or with forward primer hPARVBreal2F (5′ TGAATTTGGAGGTGACGGAACT 3′) paired with reverse primer hPARVBreal2R (5′ CAGAAGGCCCATGAGCAGAA 3′). β-actin was used as an internal control where 0.025 μl of cDNA template was used with forward primer (β-actinRTF, 5′ CCT GGC ACC CAG CAC AAT 3′) and reverse primer (β-actinRTR, 5′ GCC GAT CCA CAC GGA GTA CT 3′. Optimal PCR conditions were determined by performing primer matrix reactions and generating standard curves for both PARVB and β-actin. PCR reactions were performed in triplicate and the relative expression level of PARVB mRNA in normal and tumor was calculated by normalizing to β-actin mRNA expression levels using the comparative C_{T} (ΔΔC_{T}) method, where CT represents the cycle number at which the amplification reaches a threshold level chosen to lie in the exponential phase of all PCR reactions. Relative expression levels were calculated using the formula 2^{-ΔΔ}T, where ΔC_{T} represents the difference between the average PARVB C_{T} value and the average β-actin C_{T} value within a given tissue. ΔΔC_{T} represents the difference between the ΔC_{T} values for a matched normal and tumor pair. The normalized PARVB expression level for normal mammary gland was set to 1. Data were analyzed using ABI PRISM® 7000 sequence detection system software (Applied Biosystems).

RT-PCR was performed to amplify PARVB products from 1 μg of total RNA extracted by Trizol method from transfectant and parental cells. RT-PCR products were resolved by 1% agarose gel electrophoresis.

The sequences of the primers used were:

| | |
|---|---|
| ParvB1: | 5′-ATGAAGAAGGACGAGTCGTTCCTG-3′ |
| | (forward) |
| | 5′-TCACTCCACGTTCTTGTACTTGGTGAA-3′ |
| | (reverse) |
| ParvB2: | 5′-ATGTCCTCCGCGCCGCGCTCG-3′ |
| | (forward) |
| | 5′-TCACTCCACGTTCTTGTACTTGGTGAA-3′ |
| | (reverse) |
| ParvB3: | 5′-ATGCACCATGTGTTTAAAGATCACCAA-3′ |
| | (forward) |
| | 5′-TCACTCCACGTTCTTGTACTTGGTGAA-3′ |
| | (reverse) |
| ParvA: | 5′-TCGAATTCAATGGCCACCTCCCCGCAGAA-3′ |
| | (forward) |
| | 5′-TGCTCTAGATCACTCCACGTTACGGTACTT-3′ |
| | (reverse) |
| β-actin: | 5′-GGGACCTGACTGACTACCT-3′ |
| | (forward) |
| | 5′-CTAGAAGCATTTGCGGTGGA-3′ |
| | (reverse) |

Specimens of human breast tumors and normal mammary gland were homogenized in 1.5 ml of RIPA lysis buffer [50 mM Tris HCl pH 7.5, 150 mM NaCl, 1% Triton X-100, 0.1% SDS, 0.5% sodium deoxycholate, 5 mM EDTA, 5 mM sodium orthovanadate, and complete protease inhibitor cocktail (Roche)] per 400 mg of tissue. Lysates were incubated for 30 min (4° C.) with shaking, centrifuged, and the supernatants snap-frozen in liquid nitrogen. Protein concentration was determined using the BCA™ Protein Assay (Pierce).

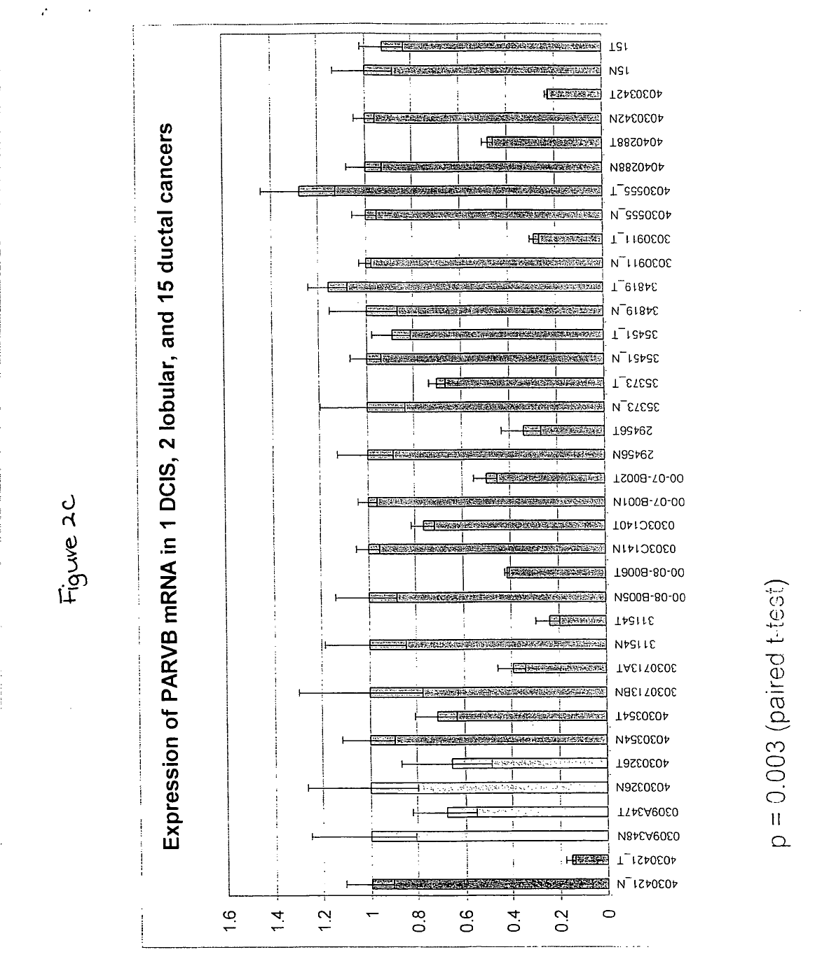

FIGS. 2A-2D demonstrate that ParvB expression is reduced in various breast cancers and cancer cell lines compared to normal.

### Analysis of Protein Expression, Signaling, and ILK Activity

Western blotting and ILK kinase assay was performed according to previously published methods (Leung-Hagesteijn et al., 2001). Affinity purified rabbit polyclonal ParvB antibody was used in primary incubations at 3 μg/ml. HRP-conjugated goat anti-mouse (Jackson ImmunoResearch) secondary antibody was incubated at 1:2000 for 1 hour at room temperature. HRP conjugated GFP antibody (Santa Cruz) and GAPDH (Ambion) were used to detect the GFP tag and as loading control, respectively. Bands were visualized with chemiluminescence substrate (ECL, Amersham). Relative band intensities were quantified using Kodak 1D2.02 software, and normalized against the GAPDH value, for each sample. Phospho-specific antibodies recognizing PKB S473 (New England Biolabs, Inc.) and GSK-3β S9 (Cell Signaling Technology) were used for analyses of the phosphorylation status of PKB and GSK-3β, as previously described (Leung-Hagesteijn et al., 2001). Relative intensity values were determined by normalizing to the most intense band, for each antibody.

### Yeast Two Hybrid Binding Assays

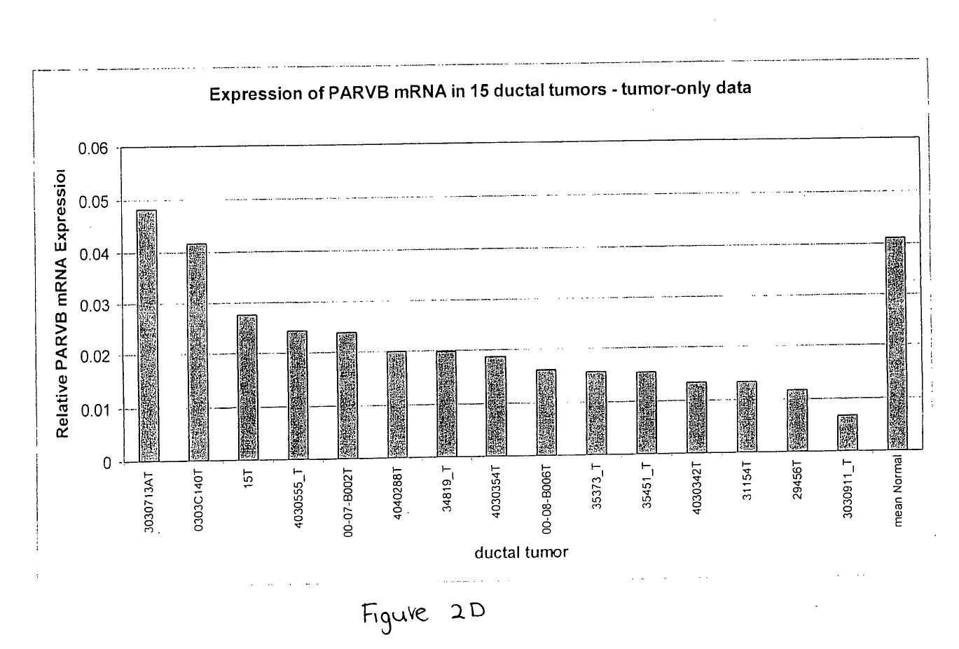

Yeast two hybrid assays were conducted using a LexA based system, and expression in EGY48 strain of Saccharomyces cerivisiae, as described previously (Hannigan et al., 1996). Full length ILK^{WT}, ILK^{E359K}, or ILKΔ^{cat} (amino acid residues 1-142, lacking the catalytic domain) cDNAs were cloned into the pEG202 bait vector, for directed interaction in yeast cells co-transformed with prey plasmid, expressing the CH2 domain of ParvB (FIG. 3C).

### Identification and Genomic Characterization of ParvB Variants

In a yeast two hybrid screen for ILK-interacting proteins, a cDNA corresponding to ParvB was identified. In order to generate ParvB expression vectors for functional studies, ParvB mRNA was amplified from a human rhabdomyosarcoma (RMS) cell line, and a ParvB cDNA sequence was cloned with an open reading frame encoding a protein sequence of 397 amino acids (‘CLINT’, GenBank accession number AAL08219). This product was subcloned into pcDNA3.1, as described in Materials and Methods. Other deposited β-parvin. cDNAs translate to protein sequences of 350 (accession number AAH46103) and 364 (accession number AAG27171) amino acid residues, indicating that a novel third ParvB isoform was identified. These isoforms are referred to as ParvB1-3, in order of increasing length, thus, the isoform identified as CLINT is hereafter called ParvB3.

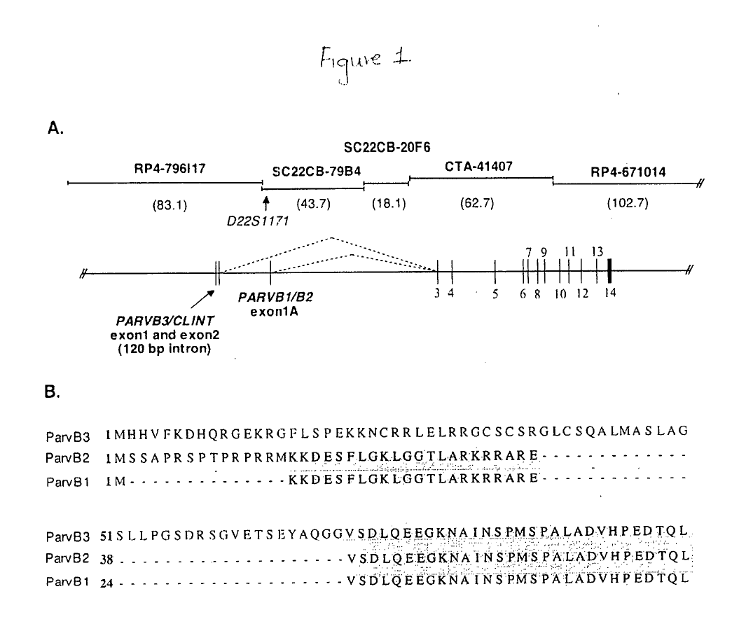

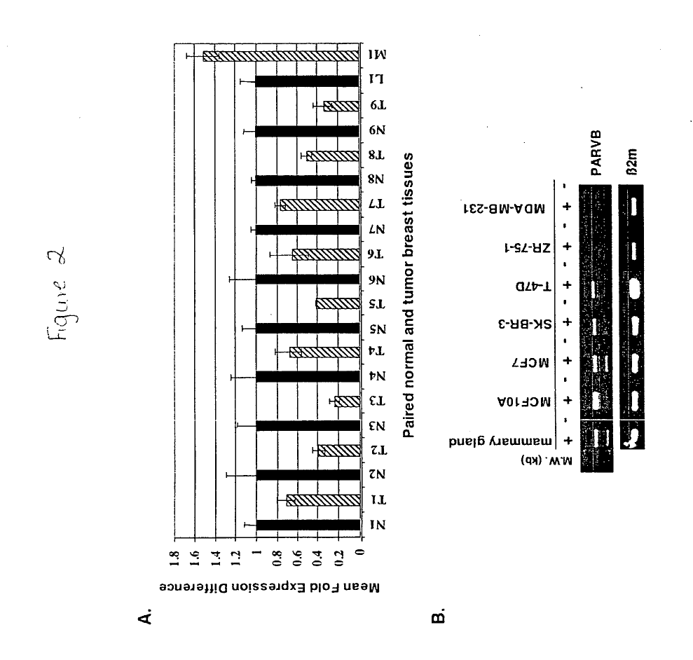

Genomic analysis indicated that ParvB3 arises from transcription of two unique 5′ exons, located on chromosome 22q13.2 (FIG. 1A). The three ParvB isoforms share an identical COOH region of 327 amino acid residues, comprised mainly of the tandem CH domains, encoded by exons 3-13 (FIG. 1A). These isoforms diverge in their NH₂-terminal regions of 23, 37 and 70 amino acids. (FIG. 1B), arising through differential splicing (ParvB3), or differential translation start site usage (ParvB1 and B2) (FIG. 1A). In order to confirm that ParvB3 is a bona fide gene product, ParvB3 specific primers were used to amplify RNA isolated from HEK293, RMS and PC3 prostate carcinoma cells. In all these cells a specific ParvB3 product was amplified, confirming expression of ParvB3 (FIG. 1C).

### ParvB mRNA Expression is Downregulated in Breast Cancer Cell Lines and Tumors

Quantitative RT-PCR was performed on RNA isolated from nine patient matched breast tumors (Bloom-Richardson Grade 2 or 3) and normal mammary tissues, using ParvB primers that would recognize all known ParvB isoforms (pan-ParvB). ParvB mRNA was downregulated by 60% or greater, in four tumors, compared to their respective matched normal mammary gland levels (FIG. 2A). In order to pursue functional studies of ParvB, ParvB expression was characterized in a number of well studied breast cancer cell lines, using the pan-ParvB primers. Two RT-PCR products were observed, of 650 and 500 basepairs in normal mammary gland, as well as MCF7, MCF10A, SK-BR-3, T-47D, MDA-MB-435, and MDA-MB-436 cells. These products were sequenced, and verified as ParvB. Interestingly, the 650 bp product was preferentially amplified in Hs578T, BT549, and BT474 cells. We did not detect any amplified ParvB product in two highly invasive breast cancer cell lines, MDA-MB-231 and ZR-75-1. RT-PCR amplification of β2-microglobulin control RNA was equally robust in all cell lines (FIG. 2B). Thus, ParvB is expressed in normal mammary gland, and our data suggest it is downregulated in advanced breast cancers, and breast cancer cell lines.

As the expression of ParvB was significantly downregulated in a number of breast tumors, the potential biological significance of this downregulation to breast cancer progression was examined. In order to pursue functional studies of ParvB in breast cancer cells, ‘empty’ pcDNA3.1, or pcDNA-ParvB3 (full length) was transfected into the poorly differentiated MDA-MB-231 line, and into the more epithelial-like MCF7 cells. Stable transfectants of each line were selected. Isoform specific RT-PCR indicated that ParvB1 and B2 transcripts were expressed in MCF7, but not MDA-MB-231 cells. Neither parental cell line expressed detectable levels of ParvB3, therefore RT-PCR readily confirmed expression of the exogenous ParvB3,gene in both, MCF7 and MDA-MB-231 transfectant lines. In addition, both cell lines expressed ParvA (FIG. 3). These lines were used to assay modulation of oncogenic cell behaviours by ParvB.

### Identification of ParvB-Deficient Breast Cancer Cell Lines

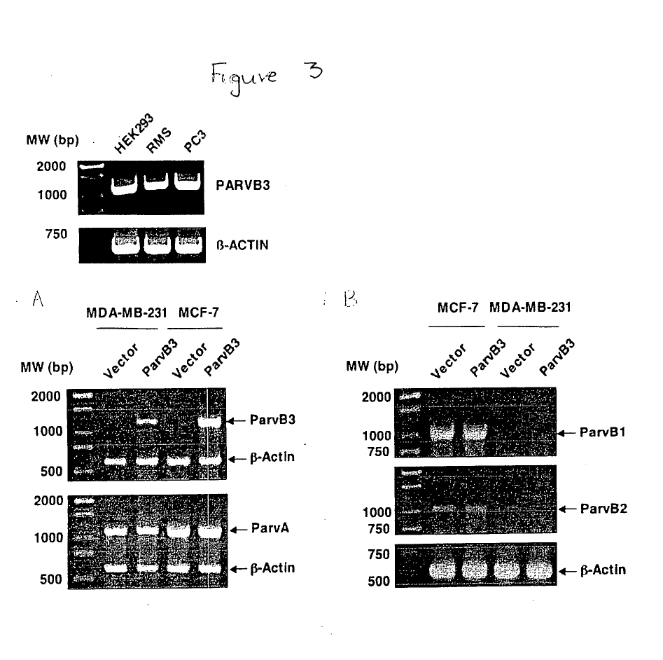

To facilitate study of ParvB protein expression, a polyclonal antibody against recombinant ParvB3 was raised, containing residues 1-142, which include the common CH1 domain (FIG. 4). HEK293 cell lysates were subjected to western blotting using antibody preparations that had been preadsorbed, either with ParvB3 or GST recombinant proteins. The affinity purified antibody recognized endogenous proteins of approximately 40kDa and 45kDa in 12% SDS-PAGE of HEK293 cell lysates, representing two ParvB isoforms (FIG. 4A). Pre-adsorption with ParvB3, but not GST, abrogated antibody recognition of these proteins. GFP-tagged ParvB3 fusion proteins were transiently expressed in HEK293 cells, in order to further test for antibody specificity. The affinity purified antibody recognized recombinant full-length ParvB3 protein, and residues 1-142, but not the COOH-terminal, residues 157-397, thus confirming its specificity (FIG. 4B, C). These results confirmed that the raised antibody detected endogenous and exogenous ParvB proteins

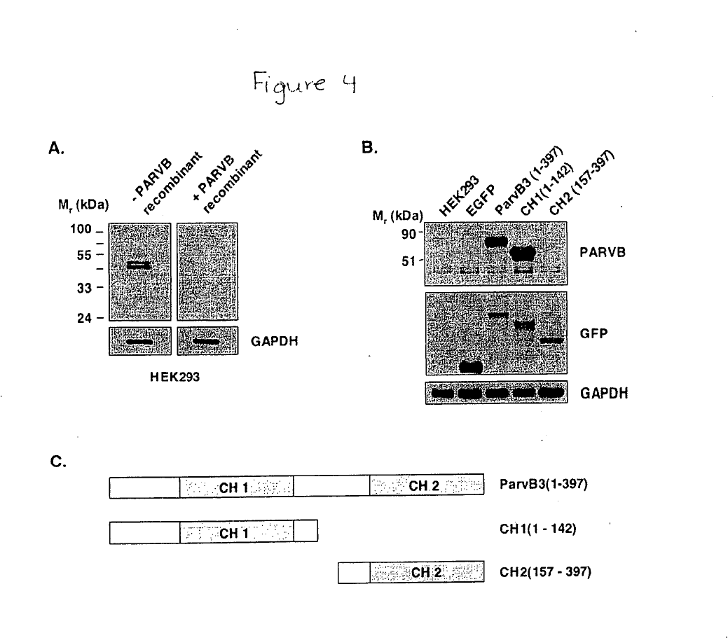

The observed downregulation of ParvB mRNA in breast tumors suggested that ParvB protein levels would be reduced as well. Using the affinity purified ParvB antibody, very low protein levels in lysates from MDA-MB-231 and MCF7 cells were detected, compared to HEK293 or PC3 prostate carcinoma cells (FIG. 5A). Interestingly, ILK protein levels were higher in the more invasive MDA-MB-231 line, compared to the MCF7 cells, consistent with its role in promoting cell invasiveness. Breast tumors were then analyzed for downregulation of ParvB protein. Patient-matched, normal and tumor tissue lysates were subjected to Western blots and ParvB signals analyzed by densitometry. ParvB protein levels were decreased by >90% in five of seven tumors, relative to levels in patient matched normal breast tissue (FIG. 5B-D). These blots were then reprobed for ILK levels. Reduced ParvB protein levels correlated with increased ILK protein in four of the seven tumor samples. To confirm that increased ILK proein correlated with increased ILK activity, three matched tumor/normal pairs were subjected to ILK immune complex kinase assays. In accord with- increased protein levels, ILK activity was elevated in these three tumors (FIG. 5C), suggesting that in some breast cancers, downregulation of ParvB contributes to upregulation of ILK signaling.

### ParvB Inhibits Anchorage Independent Growth of MCF7 and MDA-MB-231 Cells

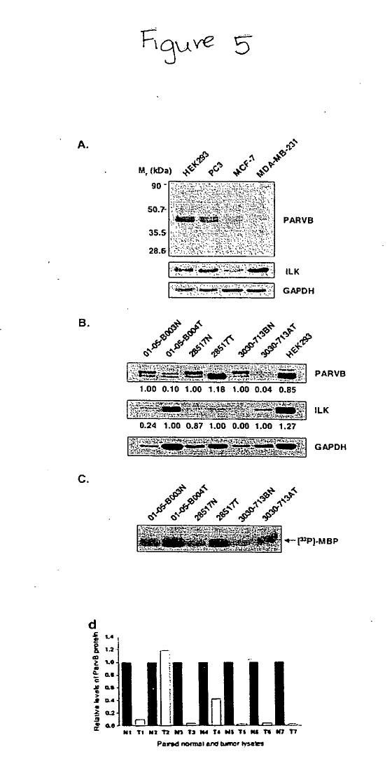

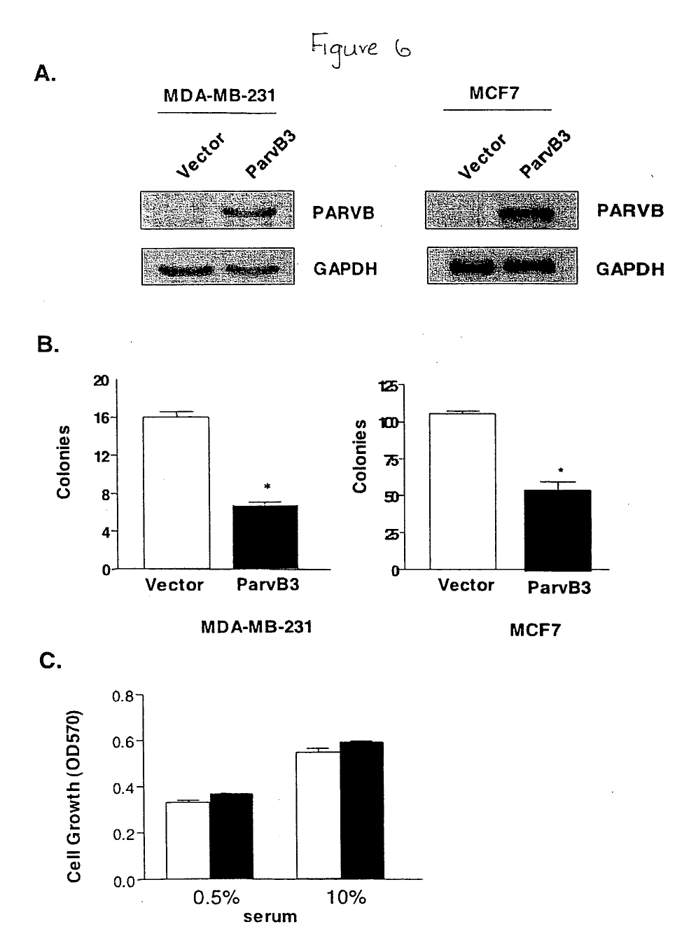

The inverse correlation between ParvB and ILK levels in breast tumors prompted the question whether ParvB can suppress the transformed cell phenotype. Moreover, as ParvB is an ILK binding protein, the potential of ParvB to inhibit oncogenic ILK signaling was examined. For these studies, the MDA-MB-231/ParvB and MCF7/ParvB transfectant cell lines were used. Lysates of stable transfectant cell lines were analysed for ParvB protein expression. Western blotting with either affinity purified ParvB antibodies (FIG. 6A), or anti-myc (not shown), confirmed appropriate expression from the ParvB3 plasmid.

As ILK induces anchorage independent growth (Hannigan et al., 1996), ParvB effects on anchorage independent growth of MDA-MB-231 and MCF7 cells were examined. Compared with vector controls, both the MDA/ParvB3 and MCF7/ParvB3 cell lines demonstrated significant suppression of colony formation in soft agar (>60% inhibition, FIG. 6B). It was then determined whether the growth suppressive effect is specific to anchorage independent growth. MTT dye assays indicated that proliferation of adherent MDA/ParvB cultures was not inhibited (FIG. 6C), nor did the cell cycle profile of adherent MDA/ParvB3 cells differ from the vector controls (Table 1). It was concluded that ParvB does not affect the intrinsic proliferative capacity of these cells. These results indicate that ParvB suppresses anchorage independent growth of both MDA-MB-231 and MCF7 cell lines.

TABLE 1
ParvB3 does not effect MDA-MB-231 Cell Cycle Distribution

| | Cell cycle | | |
|---|---|---|---|
| | distribution (%) | | |
| | G₀/G₁ | S | G₂/M |
| Vector | 75.1 | 13.7 | 10.6 |
| ParvB3 | 72.9 | 13.8 | 12.8 |

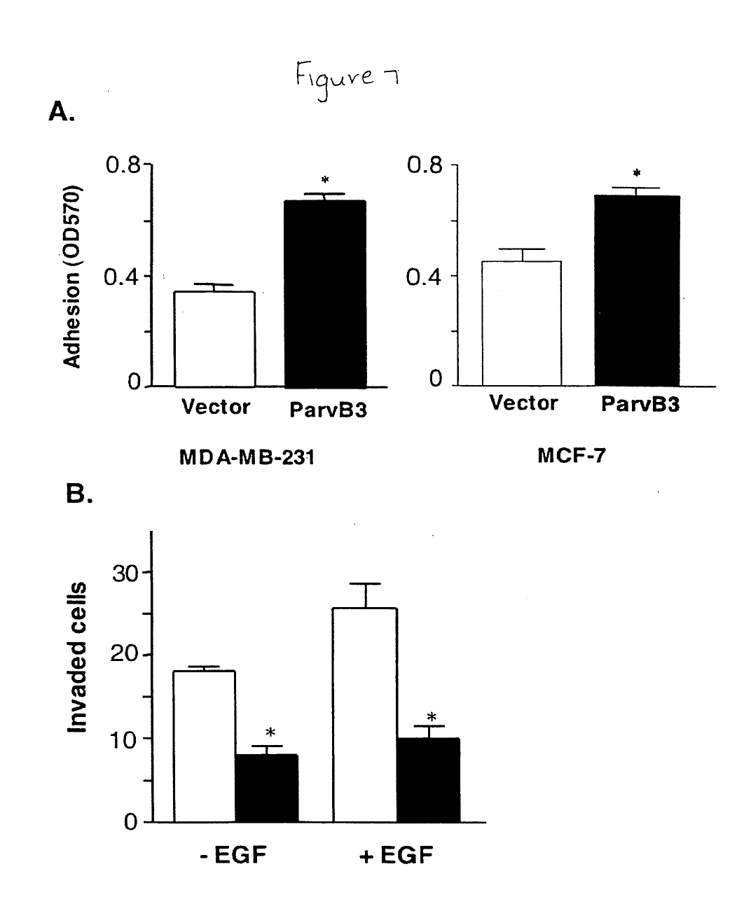

It was originally reported that expression of ILK in epithelial cells decreased cell adhesion to extracellular matrix (ECM) proteins (Hannigan et al., 1996), and ILK has also been shown to stimulate ECM invasion by epithelial cells (Troussard et al., 2000). The effects of ParvB3 expression were directly assayed on cell adhesion and ECM invasion. Firstly, vector control and ParvB3 transfectants were seeded into collagen I coated microwells, and quantified the relative proportion of cells attached to the substrate after 1 hour. ParvB expression increased MDA-MB-231 cells adhesion to collagen by about two-fold, and increased MCF7 adhesion by about 1.5 fold (FIG. 7A).

The ability of ParvB3 to suppress cell invasion through an ECM was examined. Although derived from a metastatic site, MCF7 cells retain an epithelioid morphology with intact cell-cell junctions, and are non-invasive. Conversely, MDA-MB-231 cells are highly invasive. ParvB3 effects were tested on ECM (Matrigel) invasion by MDA-MB-231 cells, using a modified Boyden chamber assay. EGF is a potent chemoattractant for the MDA-MB-231 cells (Andl et al., 2003; Price et al., 1999); thus, it was determined whether ParvB3 modulates EGF stimulated invasiveness in these cells. Cells were seeded onto Matrigel-coated membranes in the top chamber of each well, and allowed to invade for 20 hours through the Matrigel toward the bottom chamber, containing medium with or without 100 ng/ml EGF. The vector control and parental cells showed similar numbers of invaded cells, while the number of invading MDA/ParvB3 cells was suppressed by approximately 50% (FIG. 7B). EGF stimulated control cell invasiveness an additional 50%, however, it did not stimulate invasiveness of MDA/ParvB3 transfectants. These results demonstrate that ParvB3 can suppress basal and EGF-stimulated ECM invasion by breast tumor cells.

### ParvB Inhibits EGF Induced Phosphorylation of Cellular ILK Targets, PKB and GSK3β

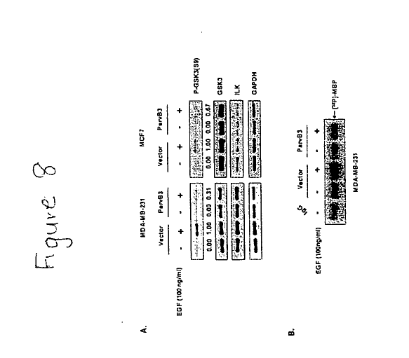

Inhibition of EGF-stimulated invasiveness suggests that ParvB inhibits signaling downstream of the EGF receptor. ILK stimulates ECM invasion of mammary epithelial cells (Troussard et al., 2000), and it is demonstrated that EGF is a potent inducer of ILK activity in kidney epithelial cells (unpublished data). Therefore, it was sought to determine whether ParvB inhibited ILK signaling in MDA/ParvB3 and MCF7/ParvB3 cells. For these studies EGF induction of GSK3β Ser9 phosphorylation was examined, by phosphospecific Western blot analysis of the MDA/ParvB3, MCF7/ParvB3, and respective vector control cells. These experiments showed that EGF-induced GSK3β Ser9 phosphorylation was suppressed by about 70% in MDA/ParvB3 cells, and by about 30% in MCF7/ParvB3 cells, compared to their respective vector controls (FIG. 8A). This quantitative difference likely reflects the substantially lower levels of ILK and EGF-induced GSK3β Ser9 phosphorylation observed in the MCF7, compared to MDA-MB-231, cells (FIGS. 5A, 8A).

It was next determined if ParvB inhibits signaling at the level of ILK activity, thereby explaining the decreased levels of GSK3β phosphorylation. EGF-induced ILK immune complex kinase activity was assayed in MDA/vector and MDA/ParvB3 cells. EGF rapidly (<15 min) induced ILK activity in the vector control cells, however, there was no detectable increase of ILK activity in EGF-treated MDA/ParvB3 cells (FIG. 8B). Thus, ParvB-mediated inhibition of GSK3β signaling appears to be due to suppression of ILK kinase activity.

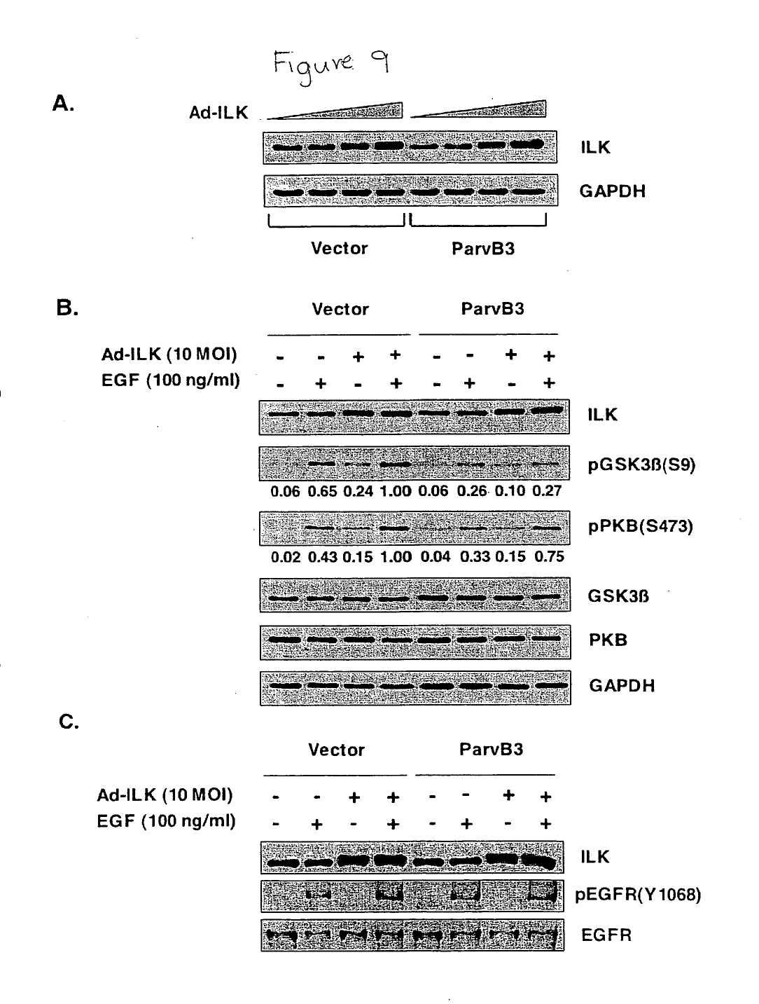

In order to test further the role of ParvB in suppressing EGF and ILK signaling, cells were infected with adenovirus expressing ILK. Infection of MDA/vector control and MBA/ParvB3 cells with Ad-ILK resulted in an approximately three-fold overexpression of ILK (FIG. 9A). ILK signaling was assayed in the Ad-ILK infected cells by Western blots, using antibodies to phospho-GSK3β(S9) and phospho-PKB(S473). Infection of vector control cells with Ad-ILK induced PKB(S473) and GSK3β(S9) phosphorylation, as expected. However, Ad-ILK or EGF-induced GSK3β(S9) phosphorylation in MDA/ParvB3 cells was only to about 30% of the levels that were induced in the vector control cells. There was a similar trend to inhibition of ILK-induced phosphorylation of PKB(S473), however, it was consistently. less pronounced than the effect on GSK3β signaling (FIG. 9B). Phosphorylation of PKB and GSK3β was effectively suppressed in Ad-ILK infected, EGF treated MDA/ParvB cells.

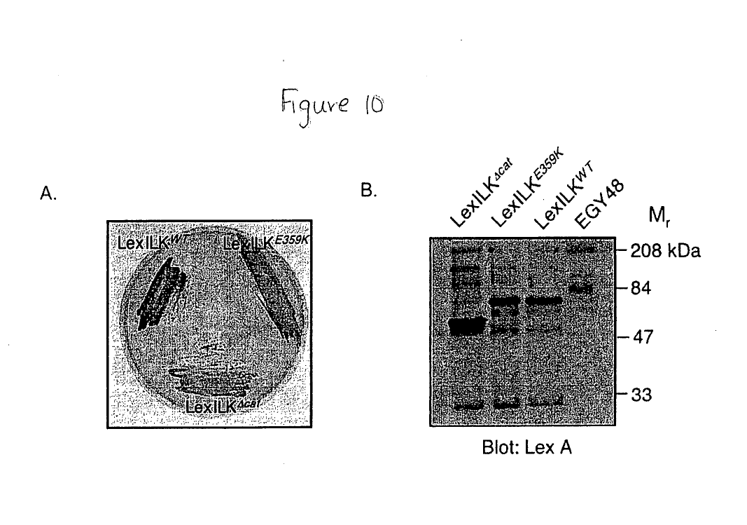

There is a possibility of integrin and EGF receptor (EGFR) crosstalk (Yu et al., 2000) suggesting that ParvB3 may be inhibiting signaling at the level of EGFR activation, and thus, EGFR expression and activation in the MDA/ParvB3 cells was examined. ParvB did not affect EGFR expression level, or EGF-induced phosphorylation at Tyr1068, indicating normal ligand-induced EGFR activation. These controls also indicated that ILK overexpression had no effect on EGFR expression, or activation (FIG. 9C). Together with data showing inhibition of ILK kinase activity (FIG. 8B), these results indicate that ParvB inhibits EGF-ILK signaling at the level of ILK. Directed yeast two hybrid assays confirmed that the ParvB3 protein interacts with ILK, requiring an intact ILK catalytic domain (FIG. 10). Thus, ParvB may inhibit ILK activity through direct association with catalytic domain residues.

TABLE ONE
397 amino acid sequence of ParvB3

MHHVFKDHQR GEKRGFLSPE KKNCRRLELR RGCSCSRGLC
SQALMASLAG
SLLPGSDRSG VETSEYAQGG VSDLQEEGKN AINSPMSPAL
ADVHPEDTQL
EENEERTMID PTSKEDPKFK ELVKVLLDWI NDVLVEERII
VKQLEEDLYD
GQVLQKLLEK LAGCKLNVAE VTQSEIGQKQ KLQTVLEAVH
DLLRPRGWAL
RWSVDSIHGK NLVAILHLLV SLAMHFRAPI RLPEHVTVQV
VVVRKREGLL
HSSHISEELT TTTEMMMGRF ERDAFDTLFD HAPDKLSVVK
KSLITFVNKH
LNKLNLEVTE LETQFADGVY LVLLMGLLED YFVPLHHFYL
TPESFDQKVH
NVSFAFELML DGGLKKPKAR PEDVVNLDLK STLRVLYNLF
TKYKNVE

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

References

Ahmed, N., Riley, C., Oliva, K., Stutt, E., G, E. R. & M, A. Q. (2003). J Pathol, 201, 229-37.
Altschul, S. F., Madden, T. L., Schaffer, A. A;, Zhang, J.; Zhang, Z., Miller, W. & Lipman, D. J. (1997). Nucleic Acids Res, 25, 3389-402.
Andl, C. D., Mizushima, T., Nakagawa, H., Oyama, K., Harada, H., Chruma, K., Herlyn, M. & Rustgi, A. K. (2003). J Biol Chem, 278, 1824-30.
Attwell, S., Mills, J., Troussard, A., Wu, C. & Dedhar, S. (2003). Mol Biol Cell.
Attwell, S., Roskelley, C. & Dedhar, S. (2000). Oncogene, 19, 3811-5.
Banuelos, S., Saraste, M. & Carugo, K. D. (1998). Structure, 6, 1419-31.
Barker, N. & Clevers, H. (2000). Bioessays, 22, 961-5.
Cairns, P., Okami, K., Halachmi, S., Halachmi, N., Esteller, M., Herman, J. G., Jen, J., Isaacs, W. B., Bova, G. S. & Sidransky, D. (1997). Cancer Res, 57, 4997-5000.
Castells, A., Gusella, J. F., Ramesh, V. & Rustgi, A. K. (2000). Cancer Res, 60, 2836-9.
Castells, A., Ino, Y., Louis, D. N., Ramesh, V., Gusella, J. F. & Rustgi, A. K. (1999). Gastroenterology, 117, 831-7.
Chung, D. H., Lee, J. I., Kook, M. C., Kim, J. R., Kim, S. H., Choi, E. Y., Park, S. H. & Song, H. G. (1998). Virchows Arch, 433, 113-7.
Coffer, P. J., Jin, J. & Woodgett, J. R. (1998). Biochem J, 335, 1-13.
D'Amico, M., Hulit, J., Amanatullah, D. F., Zafonte, B. T., Albanese, C., Bouzahzah, B., Fu, M., Augenlicht, L. H., Donehower, L. A., Takemaru, K., Moon, R. T., Davis, R., Lisanti, M. P., Shtutman, M., Zhurinsky, J., Ben-Ze'ev, A., Troussard, A. A., Dedhar, S. & Pestell, R. G. (2000). J Biol Chem, 275, 32649-57.
Dai, D. L., Makretsov, N., Campos, E. I., Huang, C., Zhou, Y., Huntsman, D., Martinka, M. & Li, G. (2003). Clin Cancer Res, 9, 4409-14.
Delcommenne, M., Tan, C., Gray, V., Rue, L., Woodgett, J. & Dedhar, S. (1998). Proc Natl Acad Sci U S A, 95, 11211-6.
Edwards, L. A., Shabbits, J. A., Bally, M. & Dedhar, S. (2004). Cancer Treat Res, 119, 59-75.
Fukuda, T., Guo, L., Shi, X. & Wu, C. (2003). J Cell Biol, 160, 1001-8.
Gimona, M. & Winder, S. J. (1998). Curr Biol, 8, R674-5.
Graff, J. R., Deddens, J. A., Konicek, B. W., Colligan, B. M., Hurst, B. M., Carter, H. W. & Carter, J. H. (2001). Clin Cancer Res, 7, 1987-91.
Hannigan, G. E., Leung-Hagesteijn, C., Fitz-Gibbon, L., Coppolino, M. G., Radeva, G., Filmus, J., Bell, J. C. & Dedhar, S. (1996). Nature, 379, 91-6.
He, T. C., Sparks, A. B., Rago, C., Hermeking, H., Zawel, L., da Costa, L. T., Morin, P. J., Vogelstein, B. & Kinzler, K. W. (1998). Science, 281, 1509-12.
Hresko, M. C., Williams, B. D. & Waterston, R. H. (1994). J Cell Biol, 124, 491-506.
Ito, R., Oue, N., Zhu, X., Yoshida, K., Nakayama, H., Yokozaki, H. & Yasui, W. (2003). Virchows Arch, 442, 118-23.
Jonsson, M., Borg, A., Nilbert; M. & Andersson, T. (2000). Eur J Cancer, 36, 242-8.
Kim, L. & Kimmel, A. R. (2000) Curr Opin Genet Dev, 10, 508-14.
Korenbaum, E., Olski, T. M. & Noegel, A. A. (2001). Gene, 279, 69-79.
Kumar, A. S., Naruszewicz, I., Wang, P., Leung-Hagesteijn, C. & Hannigan, G. E. (2004). Oncogene, 23, 3454-61.
Leslie, N. R. & Downes, C. P. (2002). Cell Signal, 14, 285-95.
Leung-Hagesteijn, C., Mahendra, A., Naruszewicz, I. & Hannigan, G. E. (2001). Embo J, 20, 2160-70.
Li, J., Yen, C., Liaw, D., Podsypanina, K., Bose, S., Wang, S. I., Puc, J., Miliaresis, C., Rodgers, L., McCombie, R., Bigner, S. H., Giovanella, B. C., Ittmann, M., Tycko, B., Hibshoosh, H., Wigler, M. H. & Parsons, R. (1997). Science, 275, 1943-7.
Lin, S. Y., Xia, W., Wang, J. C., Kwong, K. Y., Spohn, B., Wen, Y., Pestell, R. G. & Hung, M. C. (2000). Proc Natl Acad Sci U S A, 97, 4262-6.
Lin, X., Qadota, H., Moerman, D. G. & Williams, B. D. (2003). CurrBiol, 13, 922-32.
Mackinnon, A. C., Qadota, H., Norman, K. R., Moerman, D. G. & Williams, B. D. (2002). Curr Biol, 12, 787-97.
Maehama, T. & Dixon, J. E. (1999). Trends Cell Biol, 9, 125-8.
Marotta, A., Parhar, K., Owen, D., Dedhar, S. & Salh, B. (2003). Br J Cancer, 88, 1755-62.
Marotta, A., Tan, C., Gray, V., Malik, S., Gallinger, S., Sanghera, J., Dupuis, B., Owen, D., Dedhar, S. & Salh, B. (2001). Oncogene, 20, 6250-7.
Mills, G. B., Lu, Y., Fang, X., Wang, H., Eder, A., Mao, M., Swaby, R., Cheng, K. W., Stokoe, D., Siminovitch, K., Jaffe, R. & Gray, J. (2001). Semin Oncol, 28, 125-41.
Myers, M. P., Pass, I., Batty, I. H., Van der Kaay, J., Stolarov, J. P., Hemmings, B. A., Wigler, M. H., Downes, C. P. & Tonks, N. K. (1998). Proc Natl Acad Sci U S A, 95,-13513-8.
Nikolopoulos, S. N. & Turner, C. E. (2001). J Biol Chem, 276, 23499-505.
Nikolopoulos, S. N. & Turner, C. E. (2002). J Biol Chem, 277, 1568-75.
Novak, A. & Dedhar, S. (1999). Cell Mol Life Sci, 56, 523-37.
Olski, T. M., Noegel, A. A. & Korenbaum, E. (2001). J Cell Sci, 114, 525-38.
Persad, S. & Dedhar, S. (2003). Cancer Metastasis Rev, 22, 375-84.
Persad, S., Troussard, A. A., McPhee, T. R., Mulholland, D. J. & Dedhar, S. (2001). J Cell Biol, 153, 1161-74.
Price, J. T., Tiganis, T., Agarwal, A., Djakiew, D. & Thompson, E. W. (1999). Cancer Res, 59, 5475-8.
Radeva, G., Petrocelli, T., Behrend, E., Leung-Hagesteijn, C., Filmus, J., Slingerland, J. & Dedhar, S. (1997). J Biol Chem, 272, 13937-44.
Rosenberger, G., Jantke, I., Gal, A. & Kutsche, K. (2003). Hum Mol Genet, 12, 155-67.
Shtutman, M., Zhurinsky, J., Simcha, I., Albanese, C., D'Amico, M., Pestell, R. & Ben-Ze'ev, A. (1999). Proc Natl Acad Sci U S A, 96, 5522-7.
Tan, C., Costello, P., Sanghera, J., Dominguez, D., Baulida, J., de Herreros, A. G. & Dedhar, S. (2001). Oncogene, 20, 133-40.
Troussard, A. A., Costello, P., Yoganathan, T. N., Kumagai, S., Roskelley, C. D. & Dedhar, S. (2000). Oncogene, 19, 5444-52.
Tu, Y., Huang, Y., Zhang, Y., Hua, Y. & Wu, C. (2001). J Cell Biol, 153, 585-98.
White, D. E., Cardiff, R. D., Dedhar, S. & Muller, W. J. (2001). Oncogene, 20, 7064-72.
Williams, B. D. & Waterston, R. H. (1994). J Cell Biol, 124, 475-90.
Yamaji, S., Suzuki, A., Sugiyama, Y., Koide, Y., Yoshida, M., Kanamori, H., Mohri, H., Ohno, S. & Ishigatsubo, Y. (2001). J Cell Biol, 153, 1251-64.
Youssoufian, H. (1998). Biotechniques, 24, 198-200, 202.
Yu, X., Miyamoto, S. & Mekada, E. (2000). J Cell Sci, 113 ( Pt 12), 2139-47.
Zhang, Y., Guo, L., Chen, K. & Wu, C. (2002). J Biol Chem, 277, 318-26.

1. A method for the diagnosis, prognosis or prediction of breast cancer in a subject, the method comprising detecting a decrease or loss of ParvB gene expression in a tissue sample from said patient.

2. The method of claim 1, wherein said detection of a decrease or loss of ParvB expression is based on the detection of point mutations, deletions, insertions and rearrangements in the ParvB gene nucleic acid sequence.

3. The method of claim 1, wherein said detection of a decrease or loss of ParvB expression is based on the detection of a deletion on chromosome 22q13.31.

4. The method of claim 1, wherein said detection of a decrease or loss of ParvB gene expression is based on measuring transcription levels of the ParvB gene.

5. The method of claim 4, wherein said detection is done by a method selected from the group consisting of DNA/DNA hybridization, DNA/RNA hybridization, fluorescent in situ hybridization (FISH), PCR reaction and combinations thereof.

6. The method of claim 5, wherein said ParvB gene nucleic acid sequence comprises a full length nucleic acid ParvB sequence, or a functional fragment, variant or fusion product thereof.

7. The method of claim 6, wherein said ParvB nucleic acid sequence is selected from a nucleic acid sequence having at least 80% sequence, identity to said ParvB nucleic acid as measured by a BLASTN search.

8. The method of claim 7, wherein said ParvB nucleic acid sequence is selected from a nucleic acid sequence having at least 90% sequence, identity to said ParvB nucleic acid as measured by a BLASTN search.

9. The method of claim 2, wherein said ParvB nucleic acid sequence is selected from the group consisting of ParvB1, ParvB2 and ParvB3.

10. The method of claim 9, wherein said sequence is ParvB3.

11. The method of claim 1, wherein decrease or loss of ParvB gene expression results in decreased or absent ParvB protein in said tissue.

12. The method of claim 11, wherein said ParvB protein may be detected by an antibody that recognized and binds to said ParvB protein.

13. The method of claim 1, wherein said tissue is breast tissue.

14. The method of claim 1, wherein said tissue is bone tissue or lymph tissue.

15. The use of a nucleic acid encoding a ParvB tumor suppressor gene product or a functional fragment or variant thereof, or a functional fragment thereof in a method for the treatment of breast cancer in a subject, said method comprising providing a vector comprising ParvB nucleic acid by gene therapy to said subject.

16. The use of claim 15, wherein said vector is selected from the group consisting of retroviral vectors, adenovirus-associated vectors and lentiviral vectors.

17. The use of an antibody in a method for the diagnosis of breast cancer and/or prediction of likelihood of developing breast cancer in a subject, wherein said method comprises contacting an antibody that binds to a ParvB protein with a breast sample from said subject, and detecting binding of said antibody to said sample using an assay.

18. The use of claim 17, wherein said antibody is used in an assay method selected from the group consisting of western blotting and ELISA.

19. A method for producing antibodies of claim 17, comprising the steps of
administering an immunogenically effective amount of a ParvB immunogen to an animal and allowing the animal to produce antibodies to the immunogen; and
obtaining the antibodies from the animal or from a cell culture derived therefrom.

20. The method of claim 19, wherein said antibody is a substantially pure antibody which binds selectively to an antigenic determinant of a ParvB protein selected from the group consisting of ParvB1, ParvB2 and ParvB3.

21. A method for the identification of a compound which interacts with a ParvB gene product, or a functional fragment, variant or fusion product thereof, said method comprising:
incubating. a sample that is known to contain a ParvB gene product with a compound;
detecting an interaction between said ParvB gene product and said compound.

22. The method of claim 21, wherein said detecting is done by a method selected from the group consisting of phage display, yeast two-hybrid assay, co-immunoprecipitation, DNase protection assay, electrophoretic mobility shift assay and mass spectrometric analyses.

23. The method of claim 21, wherein said sample is a cell lysate or a cell culture.

24. The method of claim 23, wherein said cell is a breast cell.

25. A method for detecting a nucleic acid molecule of tumor suppressor gene ParvB, for the diagnosis or prognosis of breast cancer, the method comprising:
incubating a sample from a subject with said isolated nucleic acid molecule and determining hybridization under stringent conditions of said isolated nucleic acid molecule to a target nucleic acid molecule as a determination of presence of a nucleic acid molecule which is the ParvB tumor suppressor gene.

26. The method of claim 25, wherein such detection is done relative to a control sample.

27. A method for treating a subject suffering from breast cancer, which method comprises administering to the subject a vector that expresses a ParvB protein effective to reduce or arrest the breast cancer.

28. The method of claim 27, wherein said vector is targeted to provide intratumoral therapy.

29. A pharmaceutical composition for treating a subject suffering from breast cancer, the composition comprising a vector expressing a ParvB protein and a pharmaceutically acceptable carrier or diluent.

30. A method for suppressing the neoplastic phenotype of a breast mammary cell comprising administering to the cell an agent selected from the group consisting of
a nucleotide sequence encoding ParvB protein;
ParvB protein, fragments, polypeptides and derivatives of the polypeptides; and
an agent to stabilize ParvB protein.

31. A method for identifying compounds modulating expression of a ParvB gene comprising:
contacting a cell with a test candidate wherein the cell includes a regulatory region of a ParvB gene operably joined to a coding region; and
detecting a change in expression of the coding region.

32. A method for treating a subject having a non-transcribing or mutant ParvB gene or a lack of ParvB gene, comprising administering to the subject a therapeutically effective amount of an agent selected from the group consisting of:
(a) an isolated nucleotide sequence encoding a normal ParvB protein; and
(b) a substantially pure normal ParvB protein.

33. A method for screening for an agent useful in treating a breast cancer characterized by an increase in ILK activity, comprising screening agents for ability to promote an interaction between a ParvB gene product and ILK in a sample.

34. A method to suppress the cancerous transformed phenotype associated with increased ILK activity or non-regulated ILK activity in a cell, the method comprising upregulating the expression of ParvB in said cell.

35. A kit for identifying an agent that modulates ParvB expression, said kit comprising a vector encoding ParvB.

36. A method to identify an agent that can upregulate ParvB expression in a cell, the method comprising:
determining ParvB expression levels in a cell culture;
administering an agent to said cell culture; and
determining whether said agent has effected the ParvB expression in said cell culture.
